(19) United States
(12) Patent Application Publication
(10) Pub. No.: US 2006/0293307 A1
(43) Pub. Date: Dec. 28, 2006

(54) Oxazolidinone derivatives as antimicrobials

(76) Inventors: Anita Mehta, Gurgaon (IN); Sonali Rudra, New Delhi (IN); Ajjarapu Venkata Subrahmanya Raja Rao, Gurgaon (IN); Ashok Rattan, New Delhi (IN); Ajay Singh Yadav, Kawshmabi (IN)

Correspondence Address: RANBAXY INC., 600 COLLEGE ROAD EAST, SUITE 2100, PRINCETON, NJ, 08540, US

(21) Appl. No.: 10/523,207

(22) PCT Filed: Jul. 29, 2002
(86) PCT No.: PCT/IB02/02940

§ 371 Date: Mar. 2, 2006

(51) Int. Cl. A61K 31/551; A61K 31/496; A61K 31/498; A61K 31/454; A61K 31/473; C07D 413/14

(52) U.S. Cl. 514/218; 514/249; 514/250; 514/254.02; 514/290; 514/326; 514/299; 540/474; 540/575; 544/369; 544/350; 546/112; 546/209; 546/79

(57) ABSTRACT

The present invention relates to certain substituted phenyl oxazolidinones and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria such as multiple-resistant staphylococci, streptococci and enterococci as well as anaerobic organisms such as Bacterioides spp. and Clostridia spp. species, and acid fast organisms such as Mycobacterium tuberculosis, Mycobacterium avium and Mycobacterium spp.

### FIELD OF THE INVENTION

The present invention relates to certain substituted phenyl oxazolidinones and to processes for the synthesis of the same. This invention also relates to pharmaceutical compositions containing the compounds of the present invention as antimicrobials. The compounds are useful antimicrobial agents, effective against a number of human and veterinary pathogens, including gram-positive aerobic bacteria such as multiple-resistant staphylococci, streptococci and enterococci as well as anaerobic organisms such as Bacterioides spp. and Clostridia spp. species, and acid fast organisms such as Mycobacterium tuberculosis, Mycobacterium avium and Mycobacterium spp.

### BACKGROUND OF THE INVENTION

Increasing antibacterial resistance in Gram positive bacteria has presented a formidable treatment problem. The enterococci, although traditionally non virulent pathogens, nave been shown, when associated with Vancomycin resistance, to have an attributable mortality of approximately 40%. Staphylococcus aureus, the traditional pathogen of post operative wounds, has been resistant to Penicillin due to production of penicillinases. This resistance was overcome by the development of various penicillinase stable β lactams. But the pathogen responded by synthesizing a modified target penicillin binding protein-2′ leading to less affinity for β lactam antibiotics and a phenotype known as Methicillin Resistant S. aureus (MRSA). These strains, till recently were susceptible to Vancomycin, which inspite of its various drawbacks, has become the drug of choice for MRSA infections. Streptococcus pneumoniae is a major pathogen causing pneumonia, sinusitis and meningitis. Until very recently it was highly susceptible to penicillin. Recently though, different PBP 2′ strains with different susceptibility to penicillin have been reported from across the globe.

Oxazolidinones are a new class of synthetic antimicrobial agents which kill gram positive pathogens by inhibiting a very early stage of protein synthesis. Oxazolidinones inhibit the formation of ribosomal initiation complex involving 30S and 50S ribosomes leading to prevention of initiation complex formation. Due to their novel mechanism of action, these compounds are active against pathogens resistant to other clinically useful antibiotics.

WO 02/06278 application discloses phenyloxazolidinone derivatives as antimicrobials.

WO 93/23384 application discloses phenyloxazolidinones containing a substituted diazine moiety and their uses as antimicrobials.

WO 93/09103 application discloses substituted aryl and heteroaryl-phenyl-oxazolidinones useful as antibacterial agents.

WO90/02744 application discloses 5-indolinyl-5β-amidomethyloxazolidinones, 3-(fused ring substituted) phenyl-5β-amidomethyloxazolidinones which are useful as antibacterial agents.

European Patent Publication 352,781 discloses phenyl and pyridyl substituted phenyl oxazolidinones.

European Patent Application 312,000 discloses phenylmethyl and pyridinylmethyl substituted phenyl oxazolidinones.

U.S. Pat. No. 5,254,577 discloses nitrogen heteroaromatic rings attached to phenyloxazolidinone.

U.S. Pat. Nos. 5,547,950 and 5,700,799 also disclose the phenyl piperazinyl oxazolidinones.

J. Med. Chem. 1998; 41: 3727-3735; describes pyridine, diazene, triazene, heteroaromatic rings directly attached to the piperazinyl oxazolidinone core.

WO 98/01446 describes 6-membered heteroaryl ring containing 2 or 3 ring nitrogen atoms, attached to the piperazinyl oxazolidinyl core.

WO 98/01447 discloses pyridyl ring (optionally substituted) attached to the piperazinyl oxazolidinyl core.

U.S. Pat. No. 5,719,154 describes substituted or unsubstituted 2-pyrimidinyl, 4-pyrimidinyl, or 3-pyridazinyl rings directly attached to the piperazinyl oxazolidinyl core.

WO 00/32599 discloses phenyl oxazolidinyl as antimicrobials.

U.S. Pat. No. 5,736,545 describes azolyl piperazinyl phenyl oxazolidinones which contains azolyl ring as a five membered heterocyclic ring wherein in all the cases the piperazine nitrogen atom is attached to the carbon atom of the carbon nitrogen double bond of the five membered heterocyclic ring. The heterocycle ring contains more than one heteroatom. The five membered ring heterocycle (azolyl ring) is of the general formula: 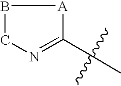
wherein A, B, and C are independently oxygen (O), nitrogen (N), sulfur (S) or carbon (C).

Other references disclosing various phenyloxazolidinones include U.S. Pat. Nos. 4,801,600 and 4,921,869; Gregory W. A., et al., J. Med. Chem., 1989; 32: 1673-81; Gregory W. A., et al., J. Med. Chem., 1990; 33: 2569-78; Wang C., et al., Tetrahedron, 1989; 45: 1323-26; Brittelli, et al., J. Med. Chem., 1992; 35: 1156; Annual reports in Medicinal Chemistry, Vol 35, pp 135-144; Bio-organic and Medicinal Chemistry Letters, 1999; 9: 2679-84; Antibacterial & Antifungal Drug Discovery & Development Summit, Strategic Research Institute, Jun. 28-29, 2001, Amsterdam, The Netherlands; Posters No. 1822, 1823, 1824, 1825, 1826, 1827, 1828, 1829, 1830, 1831, 1832, 1833, and 1834, 40^{th} Interscience Conference on Antimicrobial Agents and Chemotherapy, Sep. 17-20, (2000), Toronto, Canada; and Posters No 1023, 1040, 1041, 1042, 1043, 1044, 1045, 1046, 1047, 1048, 1049, 1050, and 1051, 41^{st} Interscience Conference on Antimicrobial Agents and Chemotherapy, Sep. 22-25, (2001), Chicago, USA.

### SUMMARY OF THE INVENTION

The objective of this invention is to synthesize, identify and profile oxazolidinone molecules which have good activity against multiply resistant gram positive pathogens like MRSA, VRE and PRSP. Some of these molecules have activity against MDR-TB and MAI strains, while others have significant activity against important anaerobic bacteria.

The compounds of the present invention are related by their substituted phenyloxazolidinone ring structure in the compounds disclosed in the publications described above except that the subject compounds have a diazine moiety attached to the phenyloxazolidinone which is further substituted by heterocyclic, aryl, substituted aryl, heteroaroamatic ring, therefore the compounds are unique and have superior antibacterial activity.

Another object of the present invention is to provide processes for the novel phenyloxazolidinones derivatives that exhibit significantly greater antibacterial activity, than available with the present compounds against multiply resistant gram positive pathogens like MRSA, VRE and PRSP against MDR-TB and MAI strains, in order to provide safe and effective treatment of bacterial infections.

In order to achieve the above-mentioned objectives and in accordance with the purpose of the invention as embodied and broadly described herein, there is provided a process for the synthesis of novel phenyloxazolidinone derivatives represented by Formula I 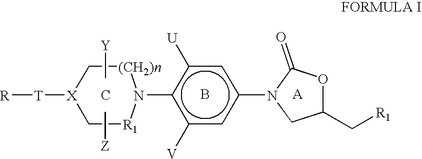
wherein
T is five membered (un)substituted heterocyclic ring with exclusively one heteroatom selected from oxygen, nitrogen and sulphur; aryl, substituted aryl, bound to the ring C. Preferred forms of T are selected from aryl and five membered heteroaryl which are further substituted by a group represented by R, wherein R is selected from the group consisting of H, CHO, C₁₋₆ alkyl, F, Cl, Br, I, —CN, COR₅,COOR₅, N(R₆,R₇), NHCOC(R₈,R₉), NHCOOR₁₀, CON (R₆,R₇), CH₂NO₂, NO₂, CH(OAc)₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄, wherein R₄ and R₅ are independently selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀=H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3;
X is C, CH, CH—S, CH—O, N, CHNR₁₁, CHCH₂NR₁₁, CCH₂NR₁₁, wherein R₁₁ is hydrogen, optionally substituted C₁₋₁₂ alkyl C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Y and Z are independently selected from hydrogen, C₁₋₆ alkyl, C₃₋₁₂ and cycloalkyl C₀₋₃ bridging groups;
U and V are independently selected from hydrogen, optionally substituted C ₁₋₆ alkyl, F, Cl, Br, C ₁₋₁₂ alkyl substituted with one or more of F, Cl, Br, I, preferably U and V are hydrogen or fluoro;
R₁ is selected from the group consisting of —NHC(═O)R₂, N(R₃,R₄), —NR₂C(═S) R₃, —NR₂C(═S)SR₃, wherein R₂ is hydrogen, C₁₋₁₂ alkyl, C₃₋₁₂ cycloalkyl, C₁₋₆ alkoxy, C₁₋₆ alkyl substituted with one or more of F, Cl, Br, I or OH; R₃,R₄ are independently selected from hydrogen, C₁₋₁₂ alkyl, C₃₋₁₂ cycloalkyl, C₁₋₆ alkoxy, C₁₋₆ alkyl substituted with one or more of F, Cl, Br, I or OH.

Preferred compounds of Formula I have R₁ as acetamide, thioacetamide or halogen substituted acetamide and the most preferred compounds in this series would be prepared as the optically pure enantiomers having the (S)-configuration according to the Cahn-Ingold-Prelog notation at C₅ of the oxazolidinone ring. The (S)-enantiomer of this series of compounds is preferred since it has two times more antibacterial activity than the corresponding racemic compound. The scope of the individual isomers and mixture of enantiomers of the structural Formula I are also covered in this invention.

Still more preferred compounds of the Formula I containing D ring as furanyl, thiophene, and pyrrolyl ring systems and further substituted by substitutions G, J and L is represented by Formula II wherein 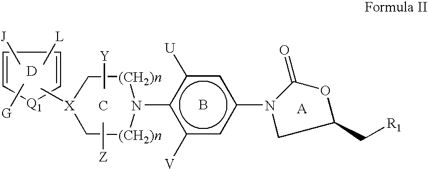
R₁ is selected from the group consisting of (1) —NHC(═O)R₂; (2) —N(R₃, R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or more of F, Cl, Br, I, OH; preferably R₁, is of the formula —NH(C═O)R₂ wherein R₂ is CH₃, CH₂F, CHF₂, CF₃, CH₂Cl. CHCl₂, CCl₃ or CHClCH₃;

U and V are independently selected from hydrogen, optionally substituted C₁₋₆ alkyl, F, Cl, Br, C₁₋₁₂ alkyl substituted with one or more of F, Cl, Br, I; preferably U and V are hydrogen and fluoro;

Y and Z are independently selected from (1) hydrogen, (2) C₁₋₆ alkyl, (3) C₃₋₁₂ cycloalkyl (4) C₀₋₃ bridging group;
X is selected from C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Q₁ is selected from O, S, NR₁₁, wherein R₁₁ is as defined above;
G, J, L are independently selected from H, C₁₋₆ alkyl, F, Cl, Br,I, —CN, CHO, COR₅,COOR₅, CH(OAc)₂, N(R₆,R₇), NHCOC(R₈,R₉,R₁₀), CON (R₆,R₇), NHCOOR₁₀, CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl.

In the more preferred compounds represented by Formula II ring C may be 6-8 membered in size and the larger rings may have either two or three carbons between each nitrogen atom, for example: 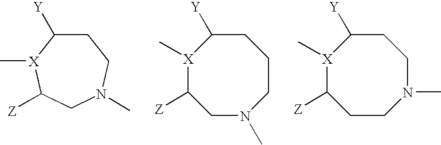
The ring C may be bridged to form a bicyclic system as shown below:

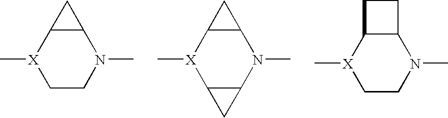

When ring C is optionally substituted at positions Y and Z with alkyl groups, cycloalkyl groups, fluoro group, carboxylic and corresponding esters, amides, substituted alkyls or bridging alkyl groups are as shown below:

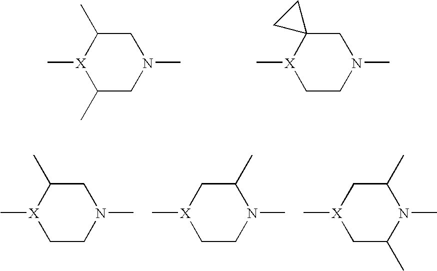

When ring C is 6 membered in size and X is —CH—(NHR₁₁), or >CCH₂NHR₁₁—, the following rings are preferred ones wherein R₁₁ is the same as defined earlier. 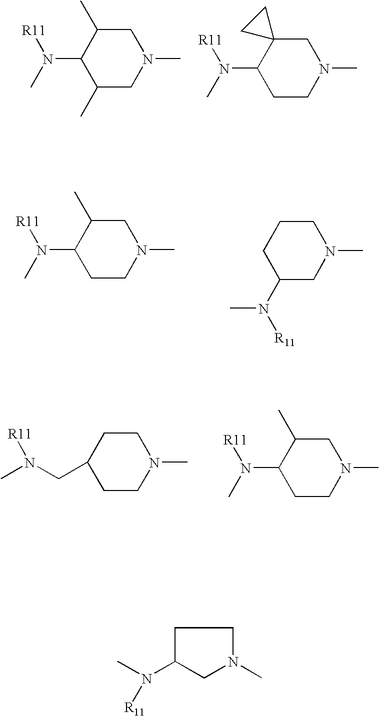
In addition to the above, ring C also includes the following structures: 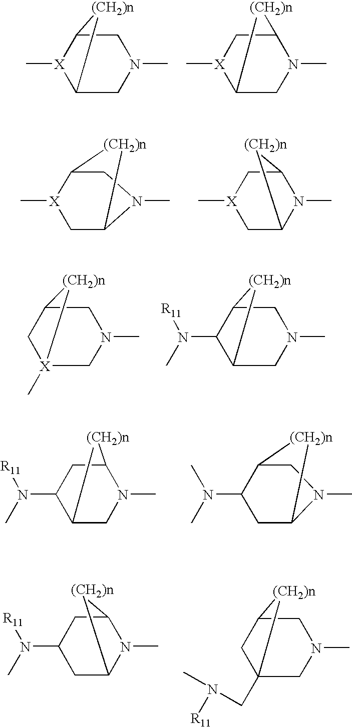
Still more preferred compounds of Formula II when Q₁=NR₁₁, is represented by Formula III 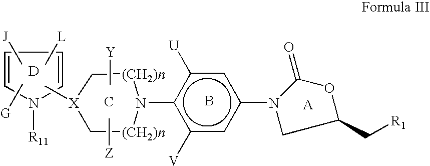
wherein
R₁ is selected from the group consisting of (1) —NHC(═O)R₂; (2) —N(R₃,R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or more of F, Cl, Br, I, OH; preferably R₁ is of the formula —NH(C═O)R₂ wherein R₂ is CH₃, CH₂F, CHF₂, CF₃, CH₂Cl_CHCl₂, CCl₃;
U and V are independently selected from hydrogen, optionally substituted C₁₋₆ alkyl, F, Cl, Br, C₁₋₁₂ alkyl substituted with one or more of F, Cl, Br, I; preferably U and V are hydrogen and fluoro.
Y and Z are independently selected from (1) hydrogen, (2) C₁₋₆ alkyl, (3) C₃₋₁₂ cycloalkyl (4) C₀₋₃ bridging group;
X is selected from C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
G, J, L are independently selected from H, C₁₋₆ alkyl, F, Cl, Br, I, —CN, COR₅,COOR₅, N(R₆,R₇), NHCOC(R₈,R₉,R₁₀), CON (R₆,R₇), NHCOOR₁₀, CH₂NO₂, NO₂, CH₂R₈, CHR₉, —CH═N—OR₁₀, —C═CH—R₅, OR₅, SR₅, C(R₉)═C(R₉)NO₂, C₁₋₁₂ alkyl substituted with one or more of F, Cl, Br, I, OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇), R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3.
More preferred G, J and L substitutions are nitro, aldehydes and halides.
Still more preferred compounds of Formula II is represented by Formula IV 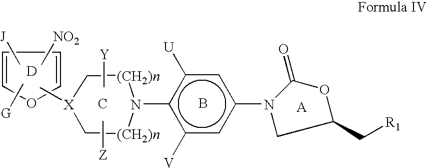
   wherein Q₁=oxygen in Formula II, and

R₁ is selected from the group consisting of (1) —NHC(═O)R₂; (2) —N(R₃,R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or more of F, Cl, Br, I, OH; preferably R₁ is of the formula —NH(C═O)R₂ wherein R₂ is CH₃, CH₂F, CHF₂, CF₃, CH₂Cl, CHCl₂, CCl₃;
U and V are independently selected from hydrogen, optionally substituted C₁₋₆ alkyl, F, Cl, Br, C₁₋₁₂ alkyl substituted with one or more of F, Cl, Br, I; preferably U and V are hydrogen and fluoro;
Y and Z are independently selected from (1) hydrogen, (2) C₁₋₆ alkyl, (3) C₃₋₁₂ ) cycloalkyl (4) C₀₋₃ bridging group;

X is selected from C, CH⁻, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
G, J, L are independently selected from H, C₁₋₆ alkyl, F, Cl, Br, I, —CN, COR₅,COOR₅, N(R₆,R₇), NHCOC(R₈,R₉,R₁₀), NHCOOR₁₀, CON⁻(R₆,R₇), CH₂NO₂, NO₂, CH₂R₈, CHR₉, —CH═N—OR₁₀, —C═CH—R₅, OR₅, SR₅, —C(R₉)═C(R₉) NO₂, C₁₋₁₂ alkyl substituted with one or more of F, Cl, Br, I, OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3.

More preferred G, J and L substitutions are nitro, aldehydes and halides.

The preferred compounds of Formula IV are as follows:
(S)—N-[[3-[3-Fluoro-4-[4-(5-nitro-2-furanyl)-1-piperazinyl]phenyl]-2-oxo-5-oxazolidinyl]methyl]acetamide

Still more preferred compounds of Formula II is represented by Formula V 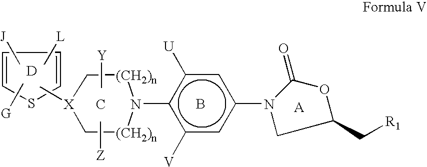
with Q₁=sulphur in Formula II, wherein
R₁ is selected from the group consisting of (1) —NHC(═O)R₂; (2) —N(R₃,R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or of more F, Cl, Br, I, OH; preferably R₁ is of the formula —NH(C═O)R₂ wherein R₂ is CH₃, CH₂F, CHF₂, CF₃, CH₂Cl. CHCl₂, CCl₃;
U and V are independently selected from hydrogen, optionally substituted C₁₋₆ alkyl F, Cl, Br, C₁₋₁₂ alkyl substituted with one or more F, Cl, Br, I; preferably U and V are hydrogen and fluoro.
Y and Z are independently selected from (1) hydrogen, (2) C₁₋₆ alkyl, (3) C₃₋₁₂ cycloalkyl (4) C₀₋₃ bridging group;
X is selected from C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
G, J, L are independently selected from H, C₁₋₆ alkyl, F, Cl, Br, I, —CN, COR₅,COOR₅, N(R₆,R₇), NHCOC(R₈,R₉,R₁₀), NHCOOR₁₀, CON (R₆,R₇), CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more F, Cl, Br I, OR₄, SR₄; wherein R₅ is selected from H, C₁₋₁₂ alkyl, C₃₋₁₂ cycloalkyl, C₁₋₆ alkoxy, C₁₋₆ alkyl substituted, with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl,
F, CI, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3.

More preferred G, J and L substitutions are nitro, aldehydes and halides.

The preferred compounds of Formula V are as follows:
(S)—N-[[3-[3-Fluoro-4-[4-(5-nitro-2-thienyl)-1-piperazinyl]phenyl]-2-oxo-5-oxazolidinyl]methyl]acetamide
(S)—N-[[3-[3-Fluoro-4-[4-(5-formyl-2-thienyl)-1-piperazinyl]phenyl]-2-oxo-5-oxazolidinyl]methyl]acetamide

The compounds of the present invention are useful as antimicrobial agents, effective against a number of human and veterinary pathogens, particularly aerobic Gram-positive bacteria, including multiply-antibiotic resistant staphylococci and streptococci, as well as anaerobic organisms such as Mycobacterium tuberculosis and other mycobacterium species.

For preparing pharmaceutical compositions from the compounds described by this invention, inert pharmaceutically acceptable carriers can be either solid or liquid. Solid form preparations include powders, tablets, dispersible granules, capsules, cachets, suppositories, and ointments. A solid carrier can be one or more substances which may also act as diluents, flavouring agents, solubilizers, lubricants, suspending agents, binders, or tablets disintegrating agents; it can also be as finely divided solid which is in admixture with the finely divided active compound. For the preparation of tablets, the active compound is mixed with carrier having the necessary binding properties in suitable proportions and compacted in the shape and size desired. The powders and tablets preferably contain from about 5 to about 70 percent of the active ingredient. Suitable solid carriers are lactose, pectin, dextrin, starch, gelatin, tragacanth, low melting wax, cocoa butter, and the like. The term “preparation” is intended to include the formulation of the active compound with encapsulating material as carrier providing a capsule in which the active component (with or without other carriers) is surrounded by carrier, which is thus in association with it. Similarly, capsules can be used as solid dosage forms suitable for oral administration.

Liquid form preparations include solutions, suspensions, and emulsions. As an example may be mentioned water or water-propylene glycol solutions for parenteral injection. Such solutions are prepared so as to be acceptable to biological systems (isotonicity, pH, etc.). Liquid preparations can also be formulated in solution in aqueous polyethylene glycol solution. Aqueous solutions suitable for oral use can be prepared by dissolving the active component in water and adding suitable colorants, flavours, stabilizing, and thickening agents as desired. Aqueous suspension suitable for oral use can be made by dispersing the finely divided active component in water with viscous material, i.e., natural or synthetic gums, resins, methyl cellulose, sodium carboxymethyl cellulose, and other well-known suspending agents.

Ointment preparations contain heavy metal salts of a compound of Formula I with a physiologically acceptable carrier. The carrier is desirably a conventional water-dispersible hydrophilic or oil-in-water carrier, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

Preferably, the pharmaceutical preparation is in unit dosage form. In such form, the preparation is subdivided into unit doses containing appropriate quantities of the active component. The, unit dosage form can be a packaged preparation, the package containing discrete capsules, powders in vials or ampoules, and ointments capsule, cachet, tablet, gel, or cream itself or it can be the appropriate number of any of these packaged forms.

The quantity of active compound in a unit dose of preparation may be varied or adjusted from less than 1 mg to several grams according to the particular application and the potency of the active ingredient.

In therapeutic use as agents for treating bacterial infections the compounds utilized in the pharmaceutical method of this invention are administered at the initial dosage of about 3 mg to about 40 mg per kilogram daily. The dosages, however, may be varied depending upon the requirements of the patient and the compound being employed. Determination of the proper dosage for a particular situation is within the smaller dosages which are less than the optimum dose. Small increments until the optimum effect under the daily dosage may be divided and administered in portions during the day if desired.

In order to achieve the above mentioned objects in accordance with the purpose of the invention as embodied and broadly described herein, there are provided process for the synthesis of compounds of Formulae I, II, III, IV and V. Pharmaceutically acceptable non-toxic acid addition salts of the compounds of the present invention of Formulae I, II, III, IV and V may be formed with inorganic or organic acids, by methods well known in the art.

The present invention also includes within its scope prodrugs of the compounds of Formulae I, II, III, IV and V. In general, such prodrugs will be functional derivatives of these compounds which readily get converted in vivo into defined compounds. Conventional procedures for the selection and preparation of suitable prodrugs are known.

The invention also includes pharmaceutically acceptable salts, enantiomers, solvates, polymorphs, diastereomers, N-oxides, metabolites in combination with pharmaceutically acceptable carrier and optionally included excipient.

Other objects and advantages of the invention will be set forth in the description which follows, and in part will be apparent from the description, or may be learned by the practice of the invention. The objects and the advantages of the invention may be released and obtained by means of the mechanism and combination pointed out in the appended claims.

### DETAILED DESCRIPTION OF THE INVENTION

The compounds of the present invention may be prepared by following the reaction sequences as depicted in the schemes defined below.

Mainly eight different amines of Formula VI 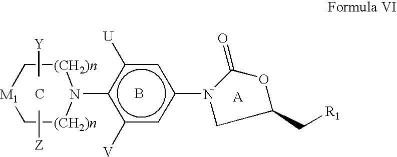
identified as ten different cores, namely
—(S)—N-[[3-[3-Fluoro-4-(N-piperazinyl)phenyl]-2-oxo-5-oxazolidinyl]methyl]acetamide (core I);
—(S)—N-[[3-[4-(N-piperazinyl)phenyl]-2-oxo-5-oxazolidinyl]methyl]acetamide (core II);
(S)—N-[[3-[3-Fluoro-4-(N-piperazinyl)phenyl]-2-oxo-5-oxazolidinyl]methyl]-2-chloropropionamide (core III);
(S)—N-[[3-[3-Fluoro-4-(N-piperazinyl)phenyl]-2-oxo-5-oxazolidinyl]methyl]-difluoroacetamide (core IV);
(S)—N-[[3-Fluoro-[4-(1-piperazinyl)-phenyl]-2-oxo-5-oxazolidinyl]-dichloroacetamide (Core V)
(S)—N-[[3-Fluoro-[4-(3-methyl-1-piperazinyl)-phenyl]-2-oxo-5-oxazolidinyl]-acetamide (Core VI)
(S)—N-[[3-[3-Fluoro-4-(N-piperazinyl)phenyl]-2-oxo-5-oxazolidinyl]-methyl]fluoroacetamide (core VII)
(S)—N-[[3-[3-Fluoro-[4-[3-(1α,5α,6α)-[6-(N-methyl)aminomethyl]-3-azabicyclo-[3.1.0] hexane]phenyl]-2-oxo-5-oxazolidinyl]methyl]acetamide (Core VII)
(S)—N-[[3-[3-Fluoro-4-(1-homopiperazenyl)phenyl]-2-oxo-5-oxazolidinyl]Methyl]acetamide (Core IX)
(S)—N-[[3-[3-Fluoro-4-(1-piperidnyl)phenyl]-2-oxo-5-oxazoldinyl]methyl]acetamide (Core X)
   were used for analoguing purposes.

Key intermediate amines of Formula VI for the analogue preparation were prepared from commercially available reagents wherein amines of Formula VI is defined as: M₁ is NH, NHR, CHNHR, —CHCH₂NHR, —CCH₂NHR wherein R is H, ethyl, methyl, isopropyl, acetyl, cyclopropyl, alkoxy, or acetyl and U, V, Y, Z, n and R₁ are as defined for Formula II.

Some amines of Formula VI are already known in the literature and are given by reference and if they have been made for the first time or by a different procedures or variation of known procedure they are described in detail in the experimental section.

Optically pure amines of Formula VI could by obtained either by one of a number of assymetric syntheses or alternatively by resolution from a racemic mixture by selective crystallization of a salt prepared, with an appropriate optically active acid such as dibenzoyl tartrate or 10-camphorsulfonic acid, followed by treatment with base to afford the optically pure amine.

The compounds of the present invention represented by general Formula I may be prepared by the method of reaction in Scheme I:

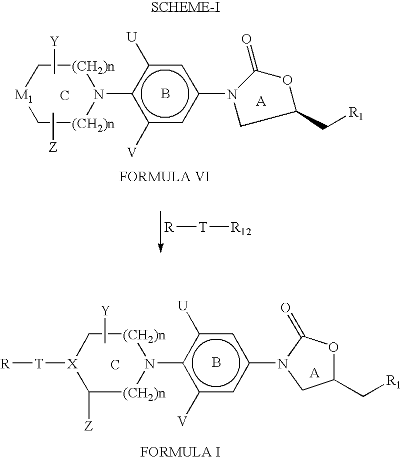

In Scheme I, the heteroaromatic group with the corresponding appendage can be introduced on the nitrogen atom of ring C of compounds of Formula VI by one of the methods described below to give Formula I, wherein R₁₂ is a suitable leaving group well known to one of ordinary skill in the art such as fluoro, chloro, bromo, iodo, SCH₃, —SO₂CH₃, —SO₂CF₃, Tos or OC₆H₅ etc., and R, T, M₁, X, R₁, U, V, Y and Z are as defined earlier.

The amine of structure of Formula VI is reacted with a heteroaromatic compound of Formula R-T-R₁₂ wherein R, T and R₁₂ are the same as defined earlier. Preferably, the reaction of Formula VI with R-T-R₁₂ is carried out in a suitable solvent in the presence of a base such as potassium carbonate, N-ethyldiisopropyl amine or dipotassium hydrogen phosphate.

The preparation of the compounds of Formula II (where heterocycle is a 5 membered ring of Formula VII wherein R₁₂ is a suitable leaving group and G, J, L, Q₁ are the same as defined earlier) is accomplished as exemplified below in Scheme II:

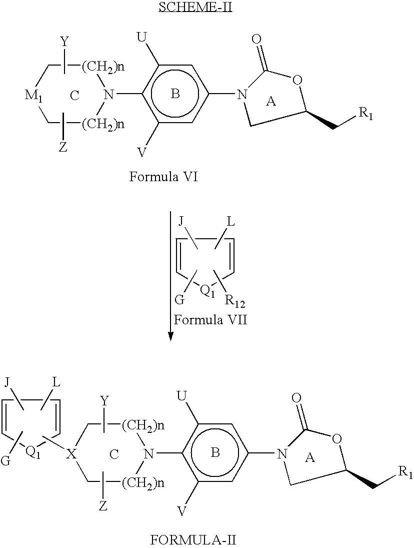

The amine of Formula VI is reacted with a heteroaromatic compound of Formula VII to give a compound of Formula II. The reaction is carried out in a suitable solvent such as dimethylformamide, dimethylacetamide, acetonitrile, dimethylsulfoxide or ethylene glycol at a suitable temperature in the range of −70° C. to 180° C. to afford compounds of Formula II. The presence of a suitable base such as triethylamine, diisopropylethylamine, potassium carbonate, sodium bicarbonate, dipotassium hydrogenphosphate is useful in some cases to improve the yield of the reaction.

Alternatively, for the preparation of compounds of Formula I, heteroaromatic compound of the Formula VII, such as 2-bromo-thiophene is reacted with the intermediate amine of Formula VI in the presence of ligands such as Palladium dibenzylidene acetone [Pd₂(dba)₃] or Pd(OAc)₂ with 2,2′-Bis-(diphenylphosphino)-1,1′-binapthyl (BINAP) and bases such as cesium carbonate or sodium t-butoxide (Ref: J. Org. Chem. 1999, 64, 6019-6022 and J. Org. Chem. 2000, 65, 1144-1157). Other ligands such as ethylenediamine or TMEDA along with bases such as cesium carbonate or potassium phosphate may also be used (Synlett, 2002, 3, 427-430).

The transformations effected are described in the experimental section. In the above synthetic methods where specific acids, bases, solvents, catalysts, oxidising agents, reducing agents etc. are mentioned, it is to be understood that the other acids, bases, solvents, catalysts, oxidising agents, reducing agents etc. may be used. Similarly, the reaction temperature and duration of the reaction may be adjusted according to the need. An illustrative list of particular compounds according to the invention and capable of being produced by the above mentioned schemes include:
(S)—N-[[3-[3-Fluoro-4-[4-(5-nitro-2-thienyl)-1-piperazinyl]phenyl]-2-oxo-5-oxazolidinyl]methyl]acetamide (Compound No. 1)
(S)—N-[[3-[3-Fluoro-4-[4-(5-formyl-2-thienyl)-1-piperazinyl]phenyl]-2-oxo-5-oxazolidinyl]methyl]acetamide (Compound No. 2)
(S)—N-[[3-[3-Fluoro-4-[4-(5-formyl-2-furyl)-1-piperazinyl]phenyl]-2-oxo-5-5 oxazolidinyl]methyl]acetamide (Compound No. 3)
(S)—N-[[3-[3-Fluoro-4-[4-(5-nitro-2-furyl)-1-piperazinyl]phenyl]-2-oxo-5-oxazolidinyl]methyl]acetamide (Compound No. 4)
(S)—N-[[3-[3-Fluoro-4-[4-{3-thienyl(2-nitro)-5-acetyloxy}methylacetate]-1-piperazinyl]phenyl]-2-oxo-5-oxazolidinyl]acetamide (Compound No. 5)
(S)—N-[[3-[4-[N-1-(5-nitro-2-thienyl)piperazinyl]-phenyl]-2-oxo-5-oxazolidinyl]-methyl]-acetamide (Compound No. 6)
(S)—N-[[3-[3-Fluoro-4-[N-1-{4-(5-nitro-2-thienyl)piperazinyl}]-phenyl]-2-oxo-5-oxazolidinyl]-methyl]-2-chloro-propionamide (Compound No. 7)
(S)—N-[[3-[3-Fluoro-4-[4-(5-nitro-2-thienyl)-1-piperazinyl]phenyl]-2-oxo-5-oxazolidinyl]methyl]difluoroacetamide (Compound No. 8)
(S)—N-[[3-[-3-Fluoro-4-[N-1-(5-nitro-2-thienyl)-piperazinyl]phenyl] -2-oxo-5-oxazolidinyl]methyl]dichloroacetamide (Compound No 9)
(S)—N-[[3-[-3-Fluoro-4-[(5-nitro-2-thienyl)-3-methyl-1-piperazinyl]phenyl]-2-oxo-5-oxazolidinyl]methyl]acetamide (Compound No. 10)
(S)—N-[[3-[3-Fluoro-4-[4-(5-nitro-2-thienyl)-1-piperazinyl]phenyl]-2-oxo-5-oxazolidinyl]methyl]fluoroacetamide (Compound No. 11)
(S)—N-[[3-[3-Fluoro-4-[3-(1α,5α,6α)-[6-{N-(5-nitro-2-thienyl)-N-methyl}aminomethyl]-3-azabicyclo-[3.1.0]hexane]phenyl]-2-oxo-5-oxazolidinyl]methyl]acetamide (Compound No 12).
(S)—N-[[3-[3-Fluoro-4-[4-(5-nitro-2-thienyl)-1-homopiperazinyl]phenyl]-2-oxo-5-oxazolidnyl]methyl]acetamide (Compound No. 13)
(S)—N-[[3-[3-Fluoro-4-[4-(5-nitro-2-furyl)-1-homopiperazinyl]phenyl]-2-oxo-5-oxazolidinyl]methyl]acetamide (Compound No. 14)
(S)—N-[[3-[3-Fluoro-4-[4-{3-thienyl(2-nitro)5-formyl}-1-piperazinyl]phenyl]-2-oxo-5-oxazolidinyl]-methyl]acetamide (Compound No. 15)
(S)—N-[[3-[3-Fluoro-4-[N-1-[4-{N-methyl-N-(5-nitro-2-furyl)}amino]-1-piperadinyl]phenyl]-2-oxo-5-oxazolidinyl]methyl]acetamide (Compound No. 16)
(S)—N-[[3-[3-Fluoro-4-[3-(1α,5α,6α)-[6-{N-(5-nitro-2-furyl)-N-methy}aminomethyl]-3-azabicyclo [3.1.0]hexane]phenyl]-2-oxo-5-oxazolidinyl]methyl]acetamide (Compound No. 17)
   Pharmacological Testing

The compounds of the invention display antibacterial activity when tested by the agar incorporation method. The following minimum inhibitory concentrations (μg/ml) were obtained for representative compounds of the invention which are given below in the following tables.

Guide to Table Abbreviations:

1) S. aureus ATCC 25923—Staphylococus aureus AXTCC 25923
2) MRSA 15187—Methicillin Resistant Staphylococcus aureus
3) Ent. faecalis ATCC 29212—Enterococcus faecium ATCC 29212
4) Ent. faecium 6A—Enterococcus faecium 6A Van®, Cipro®
5) Strep. pne. ATCC 6303—Streptococcus pneumoniae ATCC 6303
6) Strep. pyog. ATCC 19615—Streptococcus pyogenes

7) S. epidermidis—Staphylococcus epidermidis ATCC 12228

TABLE 17
MIC OF THE COMPOUNDS AGAINST 60 BACTERIAL CULTURES

| | | MIC in (μg/ml) | | | | |
|---|---|---|---|---|---|---|
| S. | | Compound | Compound | Compound | Compound | Compound |
| No. | Organisms | No. 1 | No. 2 | No. 3 | No. 4 | No 5 |
| | | 0.25 | 1 | 1 | 16 | 0.5 |
| 2 | S. aureus ATCC29213 | 0.25 | 1 | 1 | 16 | 0.25 |
| 3 | S. aureus SG 511 | 0.125 | 0.5 | 1 | 16 | 0.25 |
| 4 | S. aureus (MRSA) 15187 | 0.25 | 0.5 | 1 | 8 | 0.25 |
| 5 | S. aureus (MRSA) 21299 | 0.25 | 0.5 | 0.5 | 8 | 0.25 |
| 6 | S. aureus (MRSA) ST450 | 0.25 | 1 | 1 | 8 | 1 |
| 7 | S. aureus (MRSA 33) Cipro R | 0.25 | 0.5 | 0.5 | 8 | 0.25 |
| 8 | (MRSA) 562 | 0.25 | 0.5 | 0.5 | 16 | 0.25 |
| 9 | S. aureus (Smith 49951 | 0.25 | 0.5 | 0.5 | 16 | 0.25 |
| 10 | S. epidermidis ATCC 12228 | 0.125 | 0.5 | 0.25 | 4 | <0.06 |
| 11 | S. epidermidis (MRSE) 23760 | 0.125 | <0.25 | 0.25 | 4 | <0.06 |
| 12 | S. epidermidis 823 | 0.125 | 0.5 | 0.5 | 4 | <0.06 |
| 13 | S. epidermidis (MRSE)32965 | 0.125 | <0.25 | 0.5 | 4 | <0.06 |
| 14 | S. epidermidis 358 | 0.25 | 0.5 | 0.5 | 4 | <0.06 |
| 15 | S. haemo ATCC 29970 | 0.125 | 0.5 | 0.25 | 4 | <0.06 |
| 16 | S. warnerii ST360 | 0.125 | <0.25 | 0.25 | 8 | 0.25 |
| 17 | E. faecalis 29212 | 0.25 | <0.25 | 0.5 | 8 | 2 |
| 18 | E. faecalis 21777 | 0.25 | 0.5 | 0.5 | 8 | 1 |
| 19 | E. faecalis 5B (VRE) | 0.25 | 0.5 | NG | NG | 1 |
| 20 | E. faecalis SP 346 (VRE) | 0.25 | 0.5 | 0.5 | 8 | 2 |
| 21 | E. faecium 6A (VRE) | 0.25 | 1 | 0.5 | 8 | 2 |
| 22 | E. faecium 398(VRE) | 0.25 | 0.5 | 0.5 | 4 | 1 |
| 23 | 139 | 0.25 | 0.5 | 0.5 | 4 | 2 |
| 24 | E. durans 581 | 0.25 | 0.5 | 0.5 | 4 | 2 |
| 25 | E. coli 25922 | >16 | >16 | >16 | >16 | >8 |
| 26 | Salmonella 205 | >16 | >16 | >16 | >16 | >8 |
| 27 | K. oxytoca 49131 | >16 | >16 | >16 | >16 | >8 |
| 28 | P. aeruginosa ATCC 27853 | >16 | >16 | >16 | >16 | >8 |
| 29 | Serratia marcescens 12999 | >16 | >16 | >16 | >16 | >8 |
| 30 | Acinetobacter 9956 | >16 | >16 | >16 | >16 | >8 |
| 31 | S. pneumoniae AB-2 | 0.125 | <0.25 | <0.125 | 8 | 0.25 |
| 32 | S. pneumoniae AB-3 | 0.06 | <0.25 | 0.25 | 8 | 0.5 |
| 33 | S. pneumoniae AB4 | 0.06 | 0.5 | 0.25 | 8 | 0.5 |
| 34 | S. pneumoniae CS1221 | 0.06 | 0.5 | 0.25 | 8 | 0.5 |
| 35 | S. pneumoniae AB 10 | 0.06 | <0.25 | <0.125 | 4 | 0.25 |
| 36 | S. pneumoniae AB 31 | 0.125 | 0.5 | 0.25 | 8 | 0.5 |
| 37 | S. pneumoniae AB 14 | 0.125 | <0.25 | <0.125 | 2 | 0.5 |
| 38 | S. pneumoniae 217 | 0.125 | 0.5 | 0.25 | 4 | 0.5 |
| 39 | S. pneumoniae AB 16 | 0.125 | <0.25 | 0.25 | 8 | 0.5 |
| 40 | S. pneumoniae AB 17 | 0.06 | 0.5 | <0.125 | 4 | 0.5 |
| 41 | S. pneumoniae AB 21 | 0.125 | 0.5 | 0.25 | 8 | 0.5 |
| 42 | S. pneumoniae AB 22 | 0.125 | 0.5 | 0.25 | 8 | 0.5 |
| 43 | S. pneumoniae AB 23 | 0.125 | 0.5 | 0.25 | 8 | 0.5 |
| 44 | S. pneumoniae CS 1687 | 0.125 | 0.5 | 0.25 | 4 | 0.5 |
| 45 | S. pneumoniae AB 25 | 0.125 | <0.25 | 0.25 | 4 | 0.5 |
| 46 | S. pneumoniae AB 29 | 0.125 | <0.25 | 0.25 | 4 | 0.5 |
| 47 | S. pneumoniae AB 30 | 0.125 | 0.5 | 0.25 | 4 | 0.5 |
| 48 | S. pneumoniae ATCC 49619 | 0.06 | 0.5 | 0.25 | 4 | 0.5 |
| 49 | S. pneumoniae AB 34 | 0.25 | 0.5 | 0.25 | 4 | 0.5 |
| 50 | S. pneumoniae ATCC 6303 | 0.125 | 0.5 | 0.25 | 4 | 0.5 |
| 51 | S. pyogenes 19615 | 0.125 | 0.5 | 0.25 | 4 | 0.5 |
| 52 | S. pyogenes 25147 | 0.125 | 0.5 | 0.25 | 4 | 0.5 |
| 53 | S. pyogenes 20361 | 0.06 | 0.5 | 0.25 | 4 | 0.5 |
| 54 | S. pneumoniae 1251 | 0.125 | 0.5 | 0.25 | 4 | 0.5 |
| 55 | S pneumoniae 1294. | 0.125 | 0.5 | 0.25 | 8 | 0.5 |
| 56 | S. pneumoniae 1256 | 0.25 | <0.25 | 0.5 | 4 | 0.25 |
| 57 | S. pneumoniae 1275 | 0.06 | <0.25 | <0.125 | 2 | 0.25 |
| 58 | Moraxella M1 | 1 | 0.5 | 2 | >16 | 4 |
| 59 | Moraxella cata. M2 | 0.25 | 0.5 | 1 | >16 | 2 |
| 60 | Moraxella M6 | 0.25 | 0.5 | — | — | 2 |

| | MIC in (μg/ml) | | | | | | |
|---|---|---|---|---|---|---|---|
| S. | Compound | Compound | Compound | Compound | Compound | | |
| No. | No. 6 | No. 7 | No. 8 | No. 9 | No. 10 | Linezolid | Vancomycin |
| | 2 | >8 | 2 | 2 | 1 | 2 | 1 |
| 2 | 2 | >8 | 2 | 2 | 1 | 2 | 0.5 |
| 3 | 2 | >8 | 0.25 | 0.5 | 0.5 | 2 | 0.5 |
| 4 | 2 | >8 | 2 | 1 | 1 | 2 | 0.5 |
| 5 | 1 | >8 | 2 | 1 | 1 | 1 | 0.5 |
| 6 | 2 | >8 | 2 | 1 | 1 | 1 | 0.5 |
| 7 | 1 | >8 | 2 | 1 | 1 | 1 | 0.5 |
| 8 | 1 | >8 | 2 | 1 | 1 | 1 | 1 |
| 9 | 1 | >8 | 1 | 0.5 | 1 | 1 | 1 |
| 10 | 1 | 1 | 0.125 | 0.25 | 0.25 | <0.5 | 1 |
| 11 | 1 | 4 | 0.25 | 0.5 | 0.5 | 1 | 2 |
| 12 | 1 | 2 | 0.25 | 0.5 | 0.5 | <0.5 | 2 |
| 13 | 1 | 2 | 0.25 | 0.5 | 0.5 | 1 | 2 |
| 14 | 1 | 2 | 0.25 | 0.5 | 0.5 | 1 | 2 |
| 15 | 1 | 2 | 0.25 | 0.5 | 0.25 | <0.5 | 1 |
| 16 | 1 | 4 | 0.25 | 0.5 | 0.5 | <0.5 | 0.5 |
| 17 | 1 | >8 | 0.25 | 1 | 1 | 2 | 4 |
| 18 | 1 | >8 | 2 | 0.5 | 1 | 2 | 2 |
| 19 | NG | >8 | 2 | 0.5 | 1 | 2 | >16 |
| 20 | 1 | >8 | 0.25 | 0.5 | 1 | 2 | >16 |
| 21 | 1 | >8 | 1 | 0.5 | 1 | 2 | >16 |
| 22 | 1 | 8 | 0.25 | 0.5 | 0.5 | 1 | >16 |
| 23 | 1 | 8 | 0.25 | 0.5 | 0.5 | 1 | >16 |
| 24 | 1 | 8 | 0.25 | 0.5 | 0.5 | 1 | >16 |
| 25 | >16 | >8 | >8 | >8 | >8 | >16 | >16 |
| 26 | >16 | >8 | >8 | >8 | >8 | >16 | >16 |
| 27 | >16 | >8 | >8 | >8 | >8 | >16 | >16 |
| 28 | >16 | >8 | >8 | >8 | >8 | >16 | >16 |
| 29 | >16 | >8 | >8 | >8 | >8 | >16 | >16 |
| 30 | >16 | >8 | 8 | >8 | >8 | >16 | >16 |
| 31 | 0.25 | 0.5 | 0.125 | 0.25 | 0.5 | 0.25 | 0.5 |
| 32 | 0.25 | 0.5 | 0.125 | 0.25 | 0.5 | 0.25 | 0.5 |
| 33 | 0.25 | 0.5 | 0.125 | 0.25 | 0.5 | 0.25 | 0.25 |
| 34 | 0.25 | 0.5 | 0.125 | 0.25 | 0.5 | 0.25 | 0.25 |
| 35 | 0.25 | 0.5 | 0.125 | 0.25 | 0.25 | 0.25 | 0.25 |
| 36 | 0.5 | 1 | 0.25 | 0.25 | 0.5 | 1 | 0.25 |
| 37 | <0.125 | 1 | 0.25 | 0.25 | 0.5 | 1 | 0.25 |
| 38 | 0.25 | 1 | 0.25 | 0.25 | 0.5 | 1 | 0.5 |
| 39 | 0.5 | 1 | 0.25 | 0.25 | 0.5 | 1 | 0.25 |
| 40 | 0.25 | 1 | 0.25 | 0.25 | 0.5 | 1 | 0.25 |
| 41 | 0.5 | 1 | 0.25 | 0.25 | 0.5 | 1 | 0.25 |
| 42 | 0.25 | 1 | 0.25 | 0.25 | 0.5 | 1 | 0.25 |
| 43 | 0.5 | 1 | 0.25 | 0.25 | 0.5 | 1 | 0.25 |
| 44 | 0.5 | 2 | 0.25 | 0.5 | 0.5 | 1 | 0.25 |
| 45 | 0.5 | 0.5 | 0.25 | 0.25 | 0.5 | 1 | 0.25 |
| 46 | 0.5 | 0.5 | 0.25 | 0.25 | 0.5 | 1 | 0.25 |
| 47 | 0.5 | 0.5 | 0.25 | 0.25 | 0.25 | 1 | 0.25 |
| 48 | 0.5 | 0.5 | 0.25 | 0.25 | 0.5 | 1 | 0.25 |
| 49 | 0.5 | 4 | 0.5 | 0.5 | 1 | 2 | 0.25 |
| 50 | 0.5 | 0.5 | 0.25 | 0.25 | 0.5 | 1 | 0.25 |
| 51 | 0.5 | 2 | 0.25 | 0.5 | 0.5 | 1 | 0.5 |
| 52 | 0.5 | 2 | 0.25 | 0.5 | 0.5 | 1 | 0.5 |
| 53 | 0.5 | 2 | 0.25 | 0.5 | 0.5 | 1 | 0.5 |
| 54 | 0.25 | 1 | 0.25 | 0.25 | 0.5 | 1 | 0.25 |
| 55 | 0.5 | 2 | 0.25 | 0.5 | 0.5 | 1 | 0.5 |
| 56 | 0.25 | 0.25 | 0.125 | 0.25 | 0.25 | 1 | 0.5 |
| 57 | 0.25 | 0.25 | 0.06 | 0.25 | 0.25 | 1 | 0.5 |
| 58 | >16 | >8 | 1 | 1 | 2 | 4 | >8 |
| 59 | 8 | >8 | 1 | 1 | 2 | 4 | >8 |
| 60 | — | >8 | 1 | 1 | 2 | 4 | >8 |

TABLE 2
MIC AGAINST Haemophilus STRAINS

| | | MIC in (μg/ml) | | | | | | | |
|---|---|---|---|---|---|---|---|---|---|
| s. | | Compd. | Compd. | Compd. | Compd. | Compd. | Compd. | Compd. | Compd. |
| No. | Organisms | No. 1 | No. 2 | No. 3 | No. 5 | No. 6 | No. 7 | No. 8 | No. 9 |
| 1 | H. influenzae 35056 | 8 | 16 | 4 | >16 | 16 | >16 | >16 | >16 |
| 2 | H. influenzae ATCC | 8 | 16 | 4 | >16 | 16 | >16 | >16 | >16 |
| | 49247 | | | | | | | | |
| 3 | H. influenzae βIac | 8 | 16 | 4 | >16 | 8 | >16 | >16 | >16 |
| 4 | H. influenzae R | 8 | >16 | 8 | 16 | 16 | >16 | >16 | >16 |
| 5 | H. influenzae 23 | >16 | >16 | 8 | >16 | 16 | >16 | >16 | >16 |
| 6 | H. influenzae 49766 | 8 | >16 | 8 | >16 | 16 | >16 | >16 | >16 |
| 7 | H. influenzae 1381 | 8 | 8 | 8 | >16 | 16 | >16 | >16 | >16 |
| 8 | H. influenzae 451 | 16 | 16 | 8 | >16 | 16 | >16 | >16 | >16 |
| 9 | H. influenzae 1745 | 16 | >16 | 8 | 16 | 16 | >16 | 16 | >16 |
| 10 | H. influenzae P318 | 16 | >16 | 8 | >16 | 16 | >16 | >16 | >16 |
| 11 | H. influenzae 474 | 16 | 16 | 4 | >16 | 16 | >16 | >16 | >16 |

| | MIC in (μg/ml) | | | | | |
|---|---|---|---|---|---|---|
| s. | Compd. | | | | | |
| No. | No. 10 | Augmentin | Telithromycin | Ceftriaxone | LevoFloxaci | Linezolid |
| 1 | >16 | 2 | 2 | 0.06 | 0.015 | 8 |
| 2 | >16 | 4 | 2 | 0.125 | 0.015 | 8 |
| 3 | >16 | 1 | 2 | <0.002 | 0.008 | 8 |
| 4 | >16 | 2 | 2 | 0.004 | 0.015 | 16 |
| 5 | >16 | 1 | 2 | 0.004 | 0.015 | 16 |
| 6 | >16 | 1 | 2 | 0.008 | 0.03 | 16 |
| 7 | >16 | >16 | 2 | 0.008 | 0.015 | 16 |
| 8 | >16 | 2 | 2 | 0.008 | 0.015 | 16 |
| 9 | >16 | 2 | 1 | 0.004 | 0.03 | 16 |
| 10 | >16 | 2 | 1 | 0.015 | 0.03 | 16 |
| 11 | >16 | 2 | 2 | 0.004 | 0.015 | 16 |

TABLE 3
MIC VALUE OF COMPOUND NO. 1 AND STANDARD DRUGS AGAINST M. tuberculosis STRAINS
METHOD: AGAR DILUTION
MEDIUM: MIDDLE BROOK 7H10 + OADC
INCUBATION Temp.: 37° C.
INCUBATION PERIOD: 14-21 DAYS

| | | MIC OF STANDARD DRUGS AND COMPOUND No. 1 (μg/ml) | | | | | | |
|---|---|---|---|---|---|---|---|---|
| S. No. | STRAIN | RIF | INH | SPAR | CLA | LNZ | ETH | Compound No. 1 |
| 01.Mt- | M. tuberculosis ATCC | 0.25 | 0.125 | ≦0.125 | 32 | 1.0 | 2.0 | ≦0.125 |
| 02 Mt- | M. tuberculosis 35801 | 0.5 | 0.06 | ≦0.125 | >32 | 1.0 | 2.0 | ≦0.125 |
| 03 Mt- | M. tuberculosis ATCC | 0.125 | >32 | ≦0.125 | 32 | 1.0 | 2.0 | ≦0.125 < 0.125 |
| 04 Mt- | M. tuberculosis H₃₇Rv | 0.25 | 0.125 | ≦0.125 | 32 | 1.0 | ≦0.125 | ≦0.125 |
| 05 Mt- | M. tuberculosis SGPGI | 16 | 1.0 | 0.25 | 8.0 | 4.0 | 4.0 | 0.25 |
| 06 Mt- | M. tuberculosis SGPGI | >32 | 16 | 4.0 | 32 | >32 | >32 | 1.0 |
| 07 Mt- | M. tuberculosis SGPGI | >32 | >32 | 4.0 | 16 | >32 | >32 | 0.5 |
| 08 Mt- | M. tuberculosis M-66 | >32 | >32 | 32 | 16 | >32 | >32 | >32 |
| 09 Mt- | M. tuberculosis M-168 | 16 | 4.0 | 2.0 | 8.0 | 4.0 | >32 | 0.25 |
| 10 Mt- | M. tuberculosis M-164 | >32 | >32 | 1.0 | 16 | 32 | >32 | 0.5 |
| 11 Mt- | M. tuberculosis B-125 | 0.125 | 0.06 | ≦0.125 | 16 | 0.5 | 2.0 | ≦0.125 |
| 12 Mt- | M. tuberculosis 50 | >32 | >32 | 4.0 | 16 | >32 | 32 | 2.0 |
| 13 Mt- | M. tuberculosis V-591 | >32 | >32 | 2.0 | 32 | >32 | >32 | 0.5 |
| 14 Mt- | M. tuberculosis V-3093 | 0.125 | 0.06 | ≦0.125 | 16 | 1.0 | 2.0 | ≦0.125 |
| 15 Mt- | M. tuberculosis M-149 | >32 | 8.0 | 2.0 | 16 | 32 | >32 | 0.25 |
| 16 Mt- | M. tuberculosis PC | 4.0 | 8.0 | 2.0 | 8.0 | 32 | 32 | 0.25 |
| 17 Mt- | M. tuberculosis PC | 2.0 | 32 | 8.0 | 4.0 | 32 | 32 | 0.25 |
| 18 Mt- | M. tuberculosis PC | 0.125 | 0.25 | ≦0.125 | 32 | 0.5 | 1.0 | 0.25 |
| 19 Mt- | M. tuberculosis PC | 0.06 | 0.25 | ≦0.125 | 8.0 | 0.5 | 1.0 | ≦0.125 |
| 20 Mt- | M. tuberculosis PC | 2.0 | 16 | 8.0 | 4.0 | 32 | 16 | 0.25 |
| 21 Mt- | M. tuberculosis PC 4782 | 0.06 | 0.25 | ≦0.125 | 16 | 1.0 | 1.0 | 0.125 |
| 22 Mt- | M. tuberculosis PC | 2.0 | >32 | 4.0 | 8.0 | 32 | >32 | 0.5 |
| 23 Mt- | M. tuberculosis PC 4793 | 2.0 | >32 | 4.0 | 8.0 | 8.0 | >32 | >32 |
| 24 Mt- | M. tuberculosis H₃₇Ra | ≦0.125 | 0.25 | ≦0.12 | <0.25 | 0.5 | 2.0 | ≦0.125 |

TABLE 4
MIC VALUE OF COMPOUND NO. 1 AND STANDARD DRUGS AGAINST MAC STRAINS
METHOD: AGAR DILUTION
MEDIUM: MIDDLE BROOK 7H10 + OADC
INCUBATION Temp.: 37° C.
INCUBATION PERIOD: 14-21 DAYS

| | | MIC OF STANDARD DRUGS AND COMPOUNDS NO. 1 | | | | | | |
|---|---|---|---|---|---|---|---|---|
| S. No. | STRAIN | RIF | INH | SPAR | CLAR | LNZ | ETH | COMPOUND No. 1 |
| 01 Ma-1 | M. avium ATCC 49601 | <0.03 | >32 | 0.5 | <0.25 | 0.5 | 4.0 | <0.125 |
| 02 Ma-2 | M avium ATCC 25291 | >32 | >32 | 32 | 8.0 | 8.0 | 32 | 0.25 |
| 03 Ma-3 | M. avium ATCC 1723 | 1.0 | 32 | 4.0 | 1.0 | 16 | 16 | 0.25 |
| 04 Ma-4 | M. avium AIIMS | 4.0 | >32 | 8.0 | 1.0 | 16 | 16 | 0.25 |
| 05 Ma-6 | M avium ATCC 700897 | 1.0 | 4.0 | 2.0 | 2.0 | 16 | >32 | <0.1235 |
| 06 Mi-1 | M. intracellulare ATCC 13950 | 4.0 | >32 | 16 | 2.0 | 32 | 0.5 | 16 |
| 07 Mi-2 | M. intracellulare ATCC 35761 | 0.25 | 4.0 | 2.0 | 0.5 | 16 | >32 | <0.12 |
| 08 Mi-3 | M. intracellulare F21/12 | 4.0 | 32 | 0.125 | 1.0 | 16 | 8.0 | 0.25 |
| 09 Mi-4 | M. intracellulare B-78/3 | 0.25 | 16 | 2.0 | 1.0 | 16 | 4.0 | 0.25 |
| 10 Mai-1 | M. avium intracellulare 356/97 | 0.5 | 4.0 | 2.0 | 2.0 | 32 | 8.0 | 0.25 |
| 11 Mai-2 | M. avium intracellulare 4 | 0.25 | 2.0 | 1.0 | <0.25 | 8.0 | 4.0 | <0.125 |
| 12 Mai-4 | M. avium intracellulare 540/96 | >32 | 4.0 | 4.0 | >32 | >32 | 32 | 1.0 |
| 13 Mai-5 | M. avium intracellulare 1211/96 | 0.25 | 32 | 4.0 | 0.25 | 16 | 1.0 | 8.0 |
| 14 Mai-6 | M. avium intracellulare 926/98 | 0.25 | 4.0 | 2.0 | 1.0 | 16 | 16 | <0.12 |
| 15 Mai-7 | M. avium intracellulare 559/97 | >32 | >32 | 2.0 | 32 | 16 | >32 | 0.25 |
| 16 Mai-9 | M. avium intracellulare 18/98 | >32 | 8.0 | 2.0 | 32 | 16 | 32 | 0.25 |
| 17 Mai-10 | M. avium intracellulare 19/97 | >32 | 32 | 1.0 | 32 | 16 | 16 | 0.25 |
| 18 NTM | M. bovis ATCC 19210 | 0.125 | 0.25 | <0.12 | 32 | 1.0 | 2.0 | <0.125 |

The in vitro antibacterial activity of the compounds were demonstrated by the agar incorporation method (NCCLS M 7 and M 100-S8 documents). Briefly, the compounds were dissolved in DMSO and doubling dilution of the compounds were incorporated into Meer Hilton agar before solidification. Inoculum was prepared by suspending 4 to 5 colonies into 5 ml of normal saline solution and adjusting the turbidity to 0.5 Macfarland turbidity standard tables (1.5×10⁸ CFU/ml), after appropriate dilutions, 10⁴ CFU/spot was transfered into the surface of dried plate and incubated for 18 hours (24 hours for MRSN studies). The concentration showing no growth of the inoculated culture was recorded as the MIC. Appropriate ATCC standard strains were simultaneously tested and result recorded only when the MIC's against standard antibiotics were within the acceptable range.

The Compounds of the present invention represented by general Formula I may be prepared by the method of reaction in Scheme I. Key intermediate amines of Formula VI for the analogue preparation were prepared by the synthetic procedures described below or from commercially available reagents.

Amines already known in the literature are given by reference and if they have been made by a different procedures they are described in detail.

Mainly eight different amines of Formula VI identified as eight different cores namely
(S)—N-[[3-[3-Fluoro-4-(N-piperazinyl)phenyl]-2-oxo-5-oxazolidinyl]methyl]acetamide (core I),
(S)—N-[[3-[4-(N-piperazinyl)phenyl]-2-oxo-5-oxazolidinyl]methyl]acetamide (core II),
(S)—N-[[3-[3-Fluoro-4-(N-piperazinyl)phenyl]-2-oxo-5-oxazolidinyl]methyl]-2-chloropropionamide (core III),
(S)—N-[[3-[3-Fluoro-4-(N-piperazinyl)phenyl]-2-oxo-5-oxazolidinyl]methyl]-difluoroacetamide (core IV),
(S)—N-[[3-Fluoro-[4-(1-piperazinyl)-phenyl]-2-oxo-5-oxazolidinyl]-dichloroacetamide (Core V),
(S)—N-[[3-Fluoro-[4-(3-methyl-1-piperazinyl)-phenyl]-2-oxo-5-oxazolidinyl]-acetamide (Core VI),
(S)—N-[[3-[3-Fluoro-4-(N-piperazinyl)phenyl]-2-oxo-5-oxazolidinyl]-methyl]fluoroacetamide (core VII)
(S)—N-[[3-[3-Fluoro-[4-[3-(1α,5α,6α)-[6-1N-methyl)aminomethyl]-3-azabicyclo-[3.1.0]hexane]phenyl]-2-oxo-5-oxazolidinyl]methyl]acetamide (Core VIII)
(S)—N-[[3-[3-Fluoro-4-(1-homopiperazenyl)phenyl]-2-oxo-5-oxazolidnyl]Methyl]acetamide (Core IX)
(S)—N-[[3-[3-Fluoro-4-(1-piperidnyl)phenyl]-2-oxo-5-oxazolidinyl]methyl]acetamide (Core X) are shown in the examples given below.

Most of the compounds were characterized using NMR, IR and were purified by chromatography. Crude products were subjected to column chromatographic purification using silica gel (100-200 or 60-120 mesh) as stationary phase.

The examples mentioned below demonstrate the general synthetic procedure as well as the specific preparation for the preparation for the preferred compound. The examples are given to illustrate the details of the invention and should not be constrained to limit the scope of the present invention.

### EXAMPLE 1

### Analogues of (S)—N-[[3-[3-Fluoro-4-(N-piperazinyl)phenyl]-2-oxo-5-oxazolidinyl]methyl]acetamide(core I)

The heteroaromatic group with the corresponding appendage can be introduced on the nitrogen atom of ring C of compounds of Formula I by the methods described below:

General Procedure:

The amine of Formula VI is reacted with a heteroaromatic compound of Formula VII having R₁₂ as a suitable leaving group such as fluoro, chloro, bromo, iodo, SCH₃, —SO₂CH₃, —SO₂CF₃, Tos or OC₆H₅ etc. as defined earlier for Scheme I. Q₁, G, J and L are as defined for Formula II. The reaction is carried out in a suitable solvent such as dimethylformamide, dimethylacetamide, acetonitrile, dimethylsulfoxide or ethylene glycol at a suitable temperature in the range of −70° C. to 180° C. to afford compounds of Formula II. The presence of a suitable base such as triethylamine, diisopropylethylamine, potassium carbonate, sodium bicarbonate, dipotassium hydrogenphosphate is useful in some cases to improve the yield of the reaction.

The following compounds were made following this method:

Compound No 1: (S)—N-[[3-[3-Fluoro-4-[4-(5-nitro-2-thienyl-)-1-piperazinyl]phenyl]-2-oxo-5-oxazolidinyl]methyl]acetamide

To the (S)—N-[[3-[3-Fluoro-4-(1-piperazinyl)-phenyl]-2-oxo-5-oxazolidinyl]methyl]acetamide trifluoroacetate prepared by the method given in U.S. Pat. No 5,700,799 (4.58 mmol) in acetonitrile (40 mL), N-ethyl-diisopropylamine (5.9 g, 0.045 mol) and 5-bromo-2-nitro-thiophene (0.86 g, 5.27 mmol) were added and heated at 60° C. for 4 hrs. The reaction mixture was cooled and evaporated in vacuo. The residue was dissolved in dichloromethane (DCM) and washed with water and saturated sodium chloride solution. The organic layer was dried over sodium sulphate and evaporated in vacuo. The residue was purified by column chromatography using DCM-500 mL, 1% MeOH/DCM-200 mL, 2% MeOH/DCM-200 mL, 3% MeOH/DCM-500 mL. The product eluted in 3% MeOH/DCM. Product was sonicated in diethylether for 10 min, filtered and dried in air to get 0.493 g of the title compound. m.p. 171-174 ° C.

¹HNMR (CDCl₃): δppm 7.8 (d, 1H), 7.5 (dd, 1H), 7.11 (dd, 1H), 6.97 (t, 1H), 6.02 (m, 2H), 4.77 (m, 1H), 4.01 (t, 1H), 3.85-3.5 (m, 7H), 3.23 (m, 4H), 2.03 (s, 3H), M+1=464, M+Na=486, M+K=502, M−NO₂=418.

Compound No. 2: (S)—N-[[3-[3-Fluoro-4-[4-(5-formyl-2-thienyl-)-1-piperazinyl]phenyl]-2-oxo-5-oxazolidinyl]methyl]acetamide

To the (S)—N-[[3-[3-Fluoro-4-(1-piperazinyl)-phenyl]-2-oxo-5-oxazolidinyl]methyl]acetamide trifluoroacetate (2.28 mmol) in acetonitrile (20 mL), N-ethyl-diisopropylamine (3 g, 22.8 mmol) and 5-bromo-2-thiophenecarboxaldehyde (0.64 g, 3.4 mmol) were added and heated at 80° C. for 30 hrs. The reaction mixture was cooled and evaporated in vacuo. The residue was dissolved in dichloromethane (DCM) and washed with water and sodium chloride solution. The organic layer was dried over sodium sulphate and evaporated in vacuo. The residue was purified by column chromatography using DCM-200 ml, 1% MeOH/DCM-200 mL, 2% MeOH/DCM-400 ml, 3% MeOH/DCM-800 mL. The product eluted in 3% MeOH/DCM. The product was digested with hexane, filtered and dried in air to get 0.06 g of the title compound. m.p. 180° C. (dec), 207° C.

¹HNMR (CDCl₃): δppm 9.58 (s, 1H), 7.51 (m, 2H), 7.09 (d, 1H), 6.95 (t, 1H), 6.16 (d, 1H), 5.98 (t, 1H), 4.78 (m, 1H), 4.00 (t, 1H), 3.8-3.45 (m, 7H), 3.2 (m, 4H), 2.03 (s, 3H). M+1=447, M+Na=469, M+K=485.

Compound No. 3: (S)—N-[[3-[3-Fluoro-4-[4-(5-formyl-2-furyl)-1-piperazinyl]phenyl]-2-oxo-5-oxazolidinyl]methyl]acetamide

To the (S)—N-[[3-[3-Fluoro-4-(1-piperazinyl)-phenyl]-2-oxo-5-oxazolidinyl]methyl]acetamide (1.14 mmol) in acetonitrile (10 mL), N-ethyl-diisopropylamine (0.29 g, 2.29 mmol) and 5-bromo-2-furaldehyde (0.3 g, 1.72 mmol) were added and heated at 80° C. for 10 hrs. The reaction mixture was cooled and evaporated in vacuo. The residue was taken in dichloromethane (DCM) and washed with water and sodium chloride solution. The organic layer was dried over sodium sulphate and evaporated in vacuo. The residue was purified by column chromatography DCM-300 ml, 1% MeOH/DCM-200 mL, 2% MeOH/DCM-800 mL, 3% MeOH/DCM-800 mL. The product eluted in 3% MeOH/DCM. The product was digested with diethylether, filtered and dried in air to get 0.17 g of the title compound. m.p. 176° C.

¹HNMR(CDCl₃): δppm 9.11 (m, 1H), 7.5 (dd, 1H), 7.28 (s, 1H), 7.09 (d, 1H), 6.96 (t, 1H), 6.00 (t, 1H), 5.38 (d, 1H), 4.79 (m, 1H), 4.04 (t, 1H), 3.85-3.55 (m, 7H), 3.1 (m, 4H), 2.04 (s, 3H), M+1=431, M+Na=453, M+K=469.

Compound No. 4: (S)—N-[[3-[3-Fluoro-4-[4-(5-nitro-2-furyl)-1-piperazinyl]phenyl]-2-oxo-5-oxazolidinyl]methyl]acetamide

To the (S)—N-[[3-[3-Fluoro-4-(1-piperazinyl)-phenyl]-2-oxo-5-oxazolidinyl]methyl]acetamide hydrochloride (1.14 mmol) in N,N-dimethylformamide (10 ml), potassium carbonate (1.57 g, 11.4 mmol) was added and stirred for 15 min. 5-bromo-2-nitro-furan (0.19 g, 1.31 mmol) was added to the reaction mixture and it was stirred at room temperature for 3 hrs, when no reaction took place. Then sodium hydroxide (0.07 g) was added to the reaction mixture and stirred for 17 hrs. The reaction mixture was taken in dichloromethane (DCM) and washed with water and sodium chloride solution. The organic layer was dried over sodium sulphate and evaporated in vacuo. The residue was purified by column chromatography using DCM-200 mL, 1% MeOH/DCM-200 mL, 2% MeOH/DCM-1 L. The product eluted in 2% MeOH/DCM. The product was digested with diethylether, filtered and dried in air to get 0.32 g of the title compound. m.p. 191-204° C.

¹HNMR (CDCl₃): δppm 7.5 (m, 2H), 7.1 (d, 1H), 6.95 (t, 1H), 5.93 (t, 1H), 5.41 (d, 1H), 4.77 (m, 1H), 4.03 (t, 1H), 3.8-3.5 (m, 7H), 3.17 (m, 4H), 2.02 (s, 3H). M+1=448, M+Na=470, M+K=486, M−NO₂=486.

Compound No. 15: (S)—N-[[3-[3-Fluoro-4-[4-{3-thionyl(2-nitro)5-formyl]-1-piperazinyl]phenyl]-2-oxo-5-oxazolidinyl]methyl]acetamide.

(S)—N-[[3-[3-Fluoro-4-[N-1-[4-[3-thiophene(2-nitro)-(5-acetyloxy)methyl acetate]piperazinyl]phenyl]-2-oxo-5-oxazolidinyl]acetamide (0.16 gm, 0.0269 moles) was taken in 1N HCL (20 ml) and stirred at room temparature for 5 hrs. The reaction mixture was extracted with dichloromethane, dried on sodium sulphate and concentrated. The crude compound was purified by column chromatography by eluting with 2% methanol in dichloromethane.

Yield: 0.02 g. H NMR (DMSO): 10.0(s, 1H, CHO )8.18 (m, 1H, NH), 7.8(d, 1H, Ar—H), 7.79(d, 1H, Ar—H), 7.11-7.0, n, 2H, Ar—H), 4.76(m, 1H, CH), 4.0(t, 1H, CH), 3.8-3.3(m, 11H), 2.0(s, 3H, COCH₃).

Compound No. 5: (S)—N-[[3-[3-Fluoro-4-[4-{3-thionyl-(2-nitro)-5-acetyloxy}methylacetate]-1-piperazinyl]phenyl]-2-oxo-5-oxazolidinyl]acetamide.

(S)—N[[3-[3-Fluoro-4-(N-1-piperazinyl)phenyl]-2-oxo-5-oxazolidinyl]methyl]acetamide (0.67 gm, 1.53 moles) was dissolved in acetonitrile. To this, N-Ethyl diisopropyl amine (0.397, 3.07 moles) and 5-nitro-4-bromo-thiophene-2-acetyloxy methylacetate (0.594 gm, 2.3 moles) were added and the reaction mixture was heated at 60° C. for 6-8 hrs. The reaction mixture was concentrated. The crude compound was purified by column chromatography eluting with 2% Methanol in dichloromethane.

1HNMR (CDCl₃): δppm 7.76 (s, 1H, Ar—H), 7.53 (d, 1H, Ar—H), 7.12 (d, 1H, Ar—H), 6.97 (m, 1H, ArH), 6.91 (s, 1H, CH), 6.1 (m, 1H, NH), 4.8 (m, 1H, CH), 4.0 (m, 1H, CH), 3.78 (m, 7H, CH₂), 3.28 (m, 4H, CH₂), 2.2 (s, 6H), 2.0 (s, 3H, CH₃).

### EXAMPLE 2

### Analogues of (S)—N-[[3-[4-(1-piperazinyl)-phenyl]-2-oxo-5-oxazolidinyl]methyl]acetamide (core II)

Compound No. 6: Preparation of (S)—N-[[3-[4-[N-1-(5-nitro-2-thienyl) piperazinyl]-phenyl]-2-oxa-5-oxazolidinyl]-methyl]-acetamide.

(S)—N-[[3-[4-(1-piperazinyl)-phenyl]-2-oxo-5-oxazolidinyl]methyl]acetamide trifluoroacetate (1.076 mmol) was stirred with acetone and K₂CO₃(200 mg) for 5 minutes, then filtered and concentrated under reduced pressure. The residue was dissolved in DMSO and stirred at room temperature. To this, a stirred solution of K₂CO₃ (224 mg, 1.61 mmol) and 2-bromo-5-nitro-thiophene (246 mg, 1.18 mmol) was added at room temperature and stirred for overnight. The reaction mixture was quenched with water and extracted with DCM. The organic layer was dried over anhydrous Na₂SO₄ and concentrated under reduced pressure to get the crude product which was purified by column chromatography. (Silica gel-100-200 mesh sige) eluent: 1-2% MeOH in DCM to yield 75 mg of the title compound.

¹H NMR (CDCl₃) δppm: 7.84-7.83 (1H, s, —Ar), 7.49-7.46 (2H, d, —Ar), 7.01-6.98 (2H, d, —Ar), 6.06-6.04 (1H, s, —Ar), 5.98-5.96 (1H, m, —NH), 4.810-4.78 (1H, m, —CH), 4.10-4.04 (1H, t, —CH₂), 3.83-3.74 (3H, m, —CH₂), 3.66-3.55 (4H, s, —CH₂), 3.36-3.33 (4H, s, —CH₂), 2.06 (3H, s, —CH₃). M+1=446, M−NO₂ =400.

### EXAMPLE 3

### Analogues of (S)—N-[[3-[3-Fluoro-[4-(1-piperazinyl)-phenyl]-2-oxo-5-oxazolidinyl]-2-chloro-propionamide (Core III)

Compound No. 7: Preparation of (S)—N-[[3-[3-Fluoro-4-[N-1-{4-(5-nitro-2-thienyl)piperazinyl}]-phenyl]-2-oxo-5-oxazolidinyl]-methyl]-2-chloro-propionamide.

(S)—N-[[3-Fluoro-[4-(1-piperazinyl)-phenyl]-2-oxo-5-oxazolidinyl]-2-chloro-propionamide (WO 00/32599) (0.22 gm, 0.454 moles) was taken in acetonitrile. To this, N-ethyldiisopropylamine (0.117 gm, 0.9 moles) and 5-nitro-2-bromo-thiophene (0.13 gm, 0.681 moles) were added and the reaction mixture was heated at 60° C. for 6-8 hrs. The reaction mixture was concentrated and the crude compound was purified by column chromatography eluting with 2% MeOH in dichloromethane.

¹HNMR (CDCl₃): δppm 8.23 (m, 1H, NH), 7.8 (d, 1H, Ar—H), 7.47 (m, 1H, Ar—H), 6.98 (m, 1H, Ar—H), 6.95 (m, 1H, Ar—H), 6.06 (d, 1H, Ar—H), 4.79 (m, 1H, CH), 4.45 (m, 1H, CH), 4.0 (m, 1H, CH), 3.81 (m, 1H, CH), 3.5 (m, 6H, CH₂), 3.22 (m, 4H, NCH₂), 1.62 (d, 3H, CH₃).

### EXAMPLE 4

### Analogues of (S)—N-[[3-[3-Fluoro-4-(N-1-piperazinyl)phenyl]-2-oxo-5-oxazolidinyl]-methyl]-difluoroacetamide (core IV)

Compound No. 8: (S)—N-[[3-[3-Fluoro-4-[4-(5-nitro-2-thienyl)-1-piperazinyl]phenyl]-2-oxo-5-oxazolidinyl]methyl]difluoroacetamide

To the (S)—N-[[3-[3-Fluoro-4-(1-piperazinyl)-phenyl]-2-oxo-5-oxazolidinyl]methyl]-difluoroacetamide (1.06 mmol, prepared as described in WO 00/32599) in acetonitrile (15 mL), N-ethyl-diisopropylamine (0.27 g, 2.11 mol) and 5-bromo-2-nitro-thiophene (0.2 g, 1.21 mmol) were added and the reaction mixture was heated at 60° C. for 5 hrs. The reaction mixture was cooled and evaporated in vacuo. The residue was dissolved in dichloromethane (DCM) and washed with water and sodium chloride solution. The organic layer was dried over sodium sulphate and evaporated in vacuo. The residue was purified by column chromatography using DCM-200 mL, 1% MeOH/DCM-100 mL, 2% MeOH/DCM-300 mL. The product eluted in 2% MeOH/DCM. The product was triturated with hexane, filtered and dried in air to get 0.05 g of the title compound.

¹HNMR (CDCl₃): δppm 7.82 (d, 1H), 7.48 (dd, 1H), 7.12 (d, 1H), 6.97 (t, 1H), 6.8 (t, 1H), 6.2-5.65 (m, 2H), 4.8 (m, 1H), 4.1 (t, 1H), 3.8-3.4 (m, 7H), 3.2 (m. 4H). M+H=499, M+Na=522, M+K=538, M−NO₂ =454.

### EXAMPLE 5

### Analogues of (S)—N-[[3-[3-Fluoro-4-(1-piperazinyl)-phenyl]-2-oxo-5-oxazolidinyl]-methyl]dichloroacetamide (Core V)

Compound No 9:(S)—N-[[3-[-3-fluoro-4-[4-(5-nitro-2-thienyl)-1-piperazinyl]phenyl]-2-oxo-5-oxozolidinyl]methyl]dichloroacetamide:

(S)—N-[[3-Fluoro-[4-(1-piperazinyl)-phenyl]-2-oxo-5-oxazolidinyl]-dichloroacetamide (0.996 mmoles, WO 00/32599) was taken in acetonitrile. To this, were added N-ethyldiisopropylamine (0.35 ml, 1.984 m.moles) and 5-nitro-2-bromo-thiophene (309 mg, 1.48 m.moles). The reaction mixture was heated at 60° C. for 6-8 hrs. The reaction mixture was concentrated. The residue obtained was dissolved in ethyl acetate, washed with water. The organic layer was dried over anhydrous sodium sulphate and concentrated under reduced pressure to get the crude product. The crude compound was purified by column chromatography eluting with 2% MeOH in dichloromethane. The product was triturated with ether, filtered and dried in air to get 0.15 g of the title compound.

¹HNMR (CDCl₃)δ PPM: 8.98-8.96 (b, 1H, —NH), 7.833-7.81(d, 1H), 7.77-7.49 (dd, 1H), 7.11-7.10 (d1H), 7.039-6.97(t, 1H), 6.27(s, 1H), 6.18-6.16(d, 1H), 4.85-4.84(d, 1H), 4.13-4.7(t, 1H),3.83-3.78(t, 1H), 3.67-3.58(6H), 3.29-3.24(4H),

### EXAMPLE 6

### Analogues of (S)—N-[[3-Fluoro4-(3-methyl-1-piperazinyl)-phenyl]-2-oxo-5-oxazolidinyl]methyl]-acetamide (Core VI)

Compound No. 10: (S)—N-[[3-[-3-Fluoro4-[4-(5-nitro-2-thienyl)-3-methyl-1-piperazinyl]phenyl]-2-oxo-5-oxozolidinyl]methyl]acetamide:

(S)—N-[[3-Fluoro-[4-(3-methyl-1-piperazinyl)-phenyl]-2-oxo-5-oxazolidinyl]-acetamide (1.55 mmoles) was taken in acetonitrile. To this, were added N-ethyldiisopropylamine (1.09 ml, 6.22 m.moles) and 5-nitro-2-bromo-thiophene (485 mg, 2.33 m.moles). The reaction mixture was heated at 60° C. for 6-8 hrs. The reaction mixture was concetrated. The residue obtained was dissolved in ethyl acetate, washed with water. The organic layer was dried over anhydrous sodium sulphate and concentrated under reduced pressure to get the crude product. The crude compound was purified by column chromatography eluting with 2% MeOH in dichloromethane. The product was triturated with ether, filtered and dried in air to get 0.07 g of the title compound.

¹HNNR (CDCl₃)δ PPM: 7.817-7.801(d, 1H), 7.507-7.460(d, 1H), 7.116-7.087(d, 1H), 6.958-6.928(t, 1H), 5.972-5.956(d, 2H),4.787-4.796(t, 1H), 4.02-3.99(2H), 3.79-3.29(8H), 3.06-3.01(2H), 2.04(s, 3H), 1.05-1.48(d, 3H).

### EXAMPLE 7

### Analogues of (S)—N-[[3-3-Fluoro-4-(N-piperazinyl)phenyl]-2-oxo-5-oxazolidinyl]-methyl]fluoroacetamide (core VII)

Compound No. 11: (S)—N-[[3-[3-Fluoro-4-[4-(5-nitro-2-thienyl)-1-piperazinyl]phenyl]-2-oxo-5-oxazolidinyl]methyl]fluoroacetamide

To the (S)—N-[[3-[3-Fluoro-4-(1-piperazinyl)-phenyl]-2-oxo-5-oxazolidinyl]methyl]fluoroacetamide (0.88 mmol, prepared as described in WO 00/32599) in acetonitrile (15 mL), N-ethyl-diisopropylamine (0.23 g, 1.75 mol) and 5-bromo-2-nitro-thiophene (0.16 g, 1 mmol) were added and heated at 60° C. for 17 hrs. The reaction mixture was cooled arid evaporated in vacuo. The residue was taken in dichloromethane (DCM) and washed with water and satd. sodium chloride solution. The organic layer was dried over anhyd. sodium sulphate and evaporated in vacuo. The residue was purified by column chromatography using DCM-400 mL, 1% NeOH/DCM-200 mL, 2% MeOH/DCM-600 mL. The product eluted in 2% MeOH/DC. The product was triturated with hexane, filtered and dried in air to get 0.08 g of the title compound. m.p.=145-150° C.

¹HNMR (CDCl₃): δppm 7.8 (d, 1H), 7.48 (dd, 1H), 7.12 (dd, 1H), 6.96 (t, 1H), 6.79 (m, 1H), 6.02 (d, 1H), 4.95-4.7 (m, 3H), 4.04 (t, 1H), 3.85-3.4 (m, 7H), 3.21 (m, 4H), M+H=482, M+Na=504.

### EXAMPLE 8

### Analogues of (S)—N-[[3-[3-Fluoro-4-]3-(1α,5α,6α)-6-[(N-methyl)aminomethyl]-3-azabicyclo-[3.1.0]hexane]phenyl]-2-oxo-5-oxazolidinyl]methyl]acetamide (Core VII)

Compound No. 12 (S)—N-[[3-[3-Fluoro-4-[3-(1α,5α,6α)-[6-{N-(5-nitro-2-thienyl)-N-methyl}aminomethyl]-3-azabicyclo-[3.1.0]heexane]phenyl]-2-oxo-5-oxazolidinyl]methyl]acetamide

(S)—N-[[3-[3-Fluoro-4-[3-(1α,5α,6α)-[6-(N-methyl)aminomethyl]-3-azabicyclo-[3.1.0]hexane]phenyl]-2-oxo-5-oxazolidinyl]methyl]acetamide (0.84 mmol, prepared as described in WO 0206278) was taken in acetonitrile (20 mL). To this, were added N-ethyldiisopropylamine (0.43 g, 3.36 mmol) and 5-nitro-2-bromo-thiophene (0.262 g, 1.26 mmol) and the reaction mixture was heated at 60° C. for 48 hrs. The reaction mixture was concentrated. The residue obtained was dissolved in ethyl acetate and washed with water. The organic layer was dried over anhydrous sodium sulphate and concentrated under reduced pressure to get the crude product. The crude compound was purified by column chromatography eluting with 2% MeOH in dichloromethane. The product was triturated with ether, filtered and dried in air to get 0.12 g of the title compound.

¹HNMR (CDCl₃)δ: 7.80-7.78 (d, 1H), 7.36-7.30 (d, 1H ), 7.01-6.98 (d, 1H), 6.64-6.58(t, 1H), 6.26 (m, 1H), 5.88-5.8(d, 1H), 4.75-4.73 (m, 1H), 4.01-3.95 (t, 1H), 3.74-3.56 (5H), 3.36-3.34 (d, 2H), 3.25-3.22 (d, 2H), 3.16 (s, 3H), 2.01 (s, 3H), 1.63 (s, 2H), 1.34 (b, 1H).

Compound No.17 (S)-N-[[3-[3-Fluoro-4-[3-(1α,5α,6α)-[6-{N-(5-nitro-2-furyl)-N-methy}aminomethyl]-3-azabicyclo [3.1.0]hexane]phenyl]-2-oxo-5-oxazolidinyl]methyl]acetamide

The title compound was prepared following the process described in Example 1, Compound No. 4 by using (S)—N-[[3-[3-Fluoro-4-[3-(1α,5α,6α)-[6-{N-methy}aminomethyl]-3-azabicyclo [3.1.0]hexane]phenyl]-2-oxo-5-oxazolidinyl]methyl]acetamide.

Yeild: 0.15 g. H¹ NMR (CDCl₃): 7.5 (d, 1H, Ar—H), 7.35(d, 1H, Ar—H), 7.0 (d, 1H Ar—H), 6.6(t, 1H, Ar—H), 5.95(m, 1H, —NH), 5.33 (d, 1H, Ar—H), 4.7 (m, 1H, CH), 3.98 (1H, CH), 3.72-3.69 (m, 5H), 3.41-3.38 (d, 2H, CH₂), 3.23-3.20 (d, 2H, CH₂), 3.13 (s, 3H, —NCH₃), 2.00 (s, 3H, COCH₃), 1.64 (m, 2H), 1.27 (t, 1H).

### EXAMPLE 9

### Analogues of (S)—N-[[3-[3-Fluoro-4-(1-homopiperazenyl)phenyl]-2-oxo-5-oxazolidnyl]Methyl]acetamide (Core IX)

Compound No.13: (S)—N-[[3-[3-Fluoro-4-[4-(5-nitro-2-thienyl)-1-homopiperazenyl]phenyl]-2-oxo-5-oxazolidinyl]methyl]acetamide.

The title Compound was prepared following the process described in Example 1 using the corresponding (S)—N-[[3-[3-Fluoro-4-(1-homopiperazinyl)phenyl]-2-oxo-5-oxazolidinyl]methyl]acetamide instead of (S)—N-[[3-[3-Fluoro-4-(1-piperazinyl)phenyl]-2-oxo-5-oxazolidinyl]methyl]acetamide.

Yield: 0.22 g. ¹H NMR(CDCl₃): 7.78 (d, 1H), 7.41(dd, 1H), 7.02 (dd, 1H), 5.96 (m, 1H), 5.86(d, 1H), 4.76(m, 1H), 4.00 (t, 1H), 3.8-3.5 (m, 9H), 2.15 (m, 2H), 2.02(s, 3H). M+H=478, M+Na=500, M+K=516, M−NO2-432.

Compound No.14: (S)—N-[[3-[3-Fluoro-4-[4-(5-nitro-2-furyl)-1-homopiperazinyl]phenyl]-2-oxo-5-oxazolidinyl]methyl]acetamide.

The title Compound was prepared following the process described in Example 1, Compound No. 4 by using the corresponding (S)—N-[[3-[3-Fluoro-4-(1-homopiperazinyl)phenyl]-2-oxo-5-oxazolidinyl]methyl]acetamide instead of (S)—N-[[3-[3-Fluro-4-(1-piperazinyl)phenyl]-2-oxo-5-oxazolidinyl]methyl]acetamide.

Yield −0.24 gm. ¹H NMR (CDCl₃): 7.5(d, 1H, Ar—H), 7.38(d, 1H, Ar—H), 6.86 (t, 1H, Ar—H) 6.0 (s, 1H, NH), 5.33(1H, d, Ar—H), 4.76 (m, 1H, CH), 4.00 (t, 1H, CH), 3.76-3.69(m, 7H, CH₂), 3.65 3.5(m, 2H, CH₂), 2.11(m, 2H, CH₂), 2.02 (s, 3H, COCH₃).

### EXAMPLE 10

### (S)—N-[[3-[3-Fluoro-4-(1-piperidnyl)phenyl]-2-oxo-5-oxazolidinyl]methyl]acetamide (Core X)

Compound No. 16 (S)—N-[[3-[3-Fluoro-4-[N-1-[4-{N-methyl-N-(5-nitro-2-furyl)}amino)-1-piperadinyl]phenyl]-2-oxo-5-oxazolidinyl]methyl]acetamide.

The title compound was prepared following the process described in Example 1, Compound No.4 by using (S)—N-[[3-[3-Fluoro-4-[N-1-[4{N-methyl-N-amino-1-piperadinyl]phenyl]-2-oxo-5-oxazolidinyl]methyl]acetamide.

Yield: 0.021 g. ¹H NMR (CDCl₃): 7.5 (m, 3H, Ar—H), 7.0 (m, 2H, Ar—H), 6.0(1H, m, NH), 4.7 (m, 1H, CH), 4.1 (t, 1H, CH), 3.8-3.5(m, 9H, ), 3.0-2.8 (m, 4H,), 2.0(s, 3H, COCH₃).

While the present invention has been described in terms of its specific embodiments, certain modifications and equivalents will be apparent to those skilled in the art and are intended to be included within the scope of the present invention.

We claim:

1. A compound having the structure of Formula I: 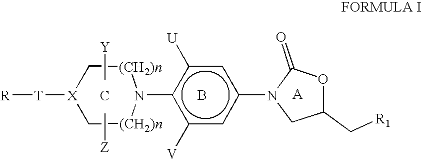
and its pharmaceutically acceptable salts, solvates, polytorphs, enantiomers, diastereomers, N-oxides, pro drugs or metabolites, wherein
T is five membered (un)substituted heterocyclic ring with exclusively one heteroatom, selected from oxygen, nitrogen and sulphur; aryl, substituted aryl, bound to the ring C including aryl and five membered heteroayl which are further substituted by a group represented by R, wherein R is selected from the group consisting of H, CHO, C₁₋₆ alkyl, F, Cl, Br, I, —CN, COR₅, COOR₅, N(R₆,R₇), NHCOC(R₈,R₉,R₁₀), NHCOOR₁₀, CON (R₆,R₇), CH₂NO₂, NO₂, CH(OAc)₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₄ and R₅ are independently selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3;
X is C, CH, CH—S, CH—O, N, CHNR₁₁, CHCH₂NR₁₁, CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Y and Z are independently selected from hydrogen, C₁₋₆ alkyl, C₃₋₁₂ and cycloalkyl C₀₋₃ bridging groups;
U and V are independently selected from hydrogen, optionally substituted C₁₋₆ alkyl, F, Cl, Br, C₁₋₁₂ alkyl substituted with one or more of F, Cl, Br, I, preferably U and V are hydrogen or fluoro;
R₁ is selected from the group consisting of —NHC(═O)R₂, N⁻(R₃, R₄), —NR₂C(═S) R₃,
—NR₂C(═S)SR₃, wherein R₂ is hydrogen, C₁₋₁₂ alkyl, C₃₋₁₂ cyoloalkyl, C₁₋₆ alkoxy, C₁₋₆ alkyl substituted with one or more of F, Cl, Br, I or OH; R₃, R₄ are independently selected from hydrogen, C₁₋₁₂ alkyl, C₃₋₁₂ cycloalkyl, C₁₋₆ alkoxy, C₁₋₆ alkyl substituted with one or more of F, Cl, Br, I or OH.

2. A compound having the structure of Formula II: 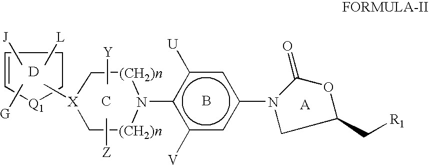
and its pharmaceutically acceptable salts, solvates, polymorphs, enantiomers, diastereomers, N-oxides, prodrugs, or metabolites, wherein
R₁ is selected from the group consisting of (1) —NHC(═O)R₂; (2) —N(R₃, R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or more of F, Cl, Br, I, OH;
U and V are independently selected from hydrogen, optionally substituted C₁₋₆ alkyl, F, Cl, Br, C₁₋₁₂ alkyl substituted with one or more of F, Cl, Br, I;
Y and Z are independently selected from (1) hydrogen, (2) C₁₋₆ alkyl, (3) C₃₋₁₂ cycloalkyl (4) C₀₋₃ bridging group.
X is selected from C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Q₁ is selected from O, S, NR₁₁, wherein R₁₁ is as defined above;
G, J, L are independently selected from H, C₁₋₆ alkyl, F, Cl, Br, I, —CN, CHO, COR₅, COOR₅, CH(OAc)₂, N(R₆,R₇), NHCOC(R₈,R₉,R₁₀), CON (R₆,R₇), NHCOOR₁₀, CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl.

3. The compound according to claim 2 wherein in Formula II, ring C is 6-8 membered in size or of larger size and the larger rings have either two or three carbons between each nitrogen atom, comprising of: 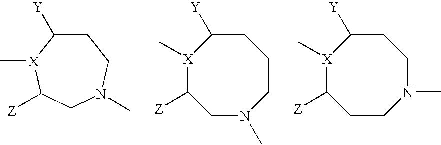
and may be bridged to form a bicyclic system as shown below, 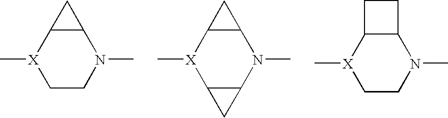
ring C optionally substituted at positions Y and Z with alkyl groups, cycloalkyl groups, fluoro group, carboxylic and corresponding esters, amides, substituted alkyls or bridging alkyl groups as shown below: 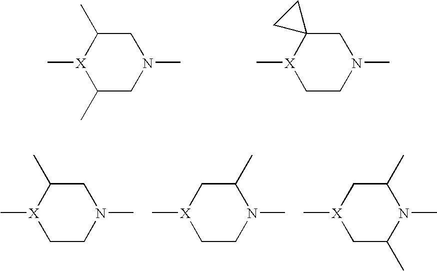
or ring C is 6 membered in size and X is —CH—(NHR₁₁), or >CCH₂NHR₁₁— which is selected from the group consisting of the following rings wherein R₁₁ is the same as defined earlier, 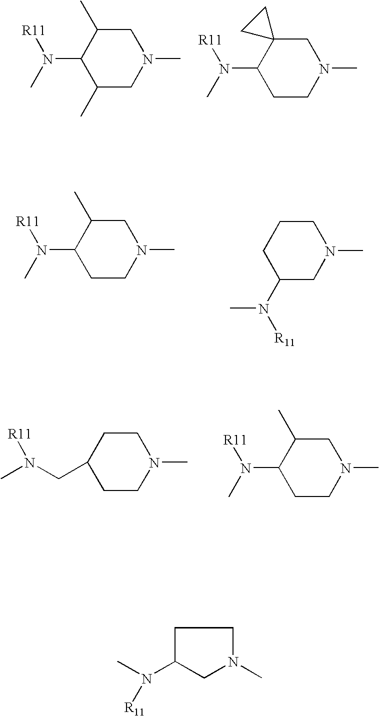
or
in addition to the above, ring C includes the following structures: 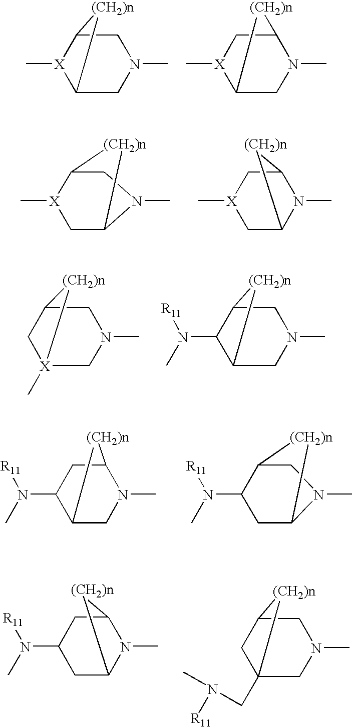
when Q₁=R₁₁, O or S, the structures are represented by Formulae III, IV and V, respectively, 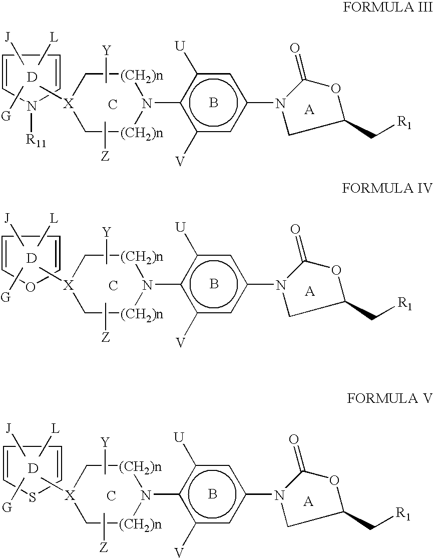
wherein R₁, R₁₁, U, V, X, Y, Z, G, J, L and n in Formula III, Formula IV and Formula V are the same as defined earlier for Formula II.

4. A compound selected from the group consisting of
(S)—N-[[3-[3-Fluoro-4-[4-(5-nitro-2-thienyl)-1-piperazinyl]phenyl]-2-oxo-5-oxazolidinyl]methyl]acetamide (Compound No.1)
(S)—N-[[3-[3-Fluoro-4-[4-(5-formyl-2-thienyl)-1-piperazinyl]phenyl]-2-oxo-5-oxazolidinyl]methyl]acetamide (Compound No.2)
(S)—N-[[3-[3-Fluoro-4-[4-(5-formyl-2-furyl)-1-piperazinyl]phenyl]-2-oxo-5-oxazolidinyl]methyl]acetamide (Compound No. 3)
(S)—N-[[3-[3-Fluoro-4-[4-(5-nitro-2-furyl)-1-piperazinyl]phenyl]-2-oxo-5-oxazolidinyl]methyl]acetamide (Compound No. 4)
(S)—N-[[3-[3-Fluoro-4-[4-{3-thienyl(2-nitro)-5-acetyloxy}methylacetate]-1-piperazinyl]phenyl]-2-oxo-5-oxazolidinyl]acetamide (Compound No. 5)
(S)—N-[[3-[4-[N-1-(5-nitro-2-thienyl) piperazinyl]-phenyl]-2-4oxa-5-oxazolidinyl]-methyl]-acetamide (Compound No. 6)
(S)—N-[[3-[3-Fluoro-4-[N-1-{4-(5-nitro-2-thienyl)piperazinyl}]-phenyl]-2-oxo-5-oxazolidinyl]-methyl]-2-chloro-propionamide (Compound No. 7)
(S)—N-[[3-[3-Fluoro-4-[4-(5-nitro-2-thienyl)-1-piperazinyl]phenyl]-2-oxo-5-oxazolidinyl]methyl]difluoroacetamide (Compound No. 8)
(S)—N-[[3-[-3-Fluoro-4-[N-1-(5-nitro-2-thienyl)-piperazinyl]phenyl]-2-oxo-5-oxozolidinyl]methyl]dichloro acetamide (Compound No 9)
(S)—N-[[3-[-3-Fluoro-4-[(5-nitro-2-thienyl)-3-methyl-1-piperazinyl]phenyl]-2-oxo-5-oxozolidinyl]methyl]acetamide (Compound No. 10)
(S)—N-[[3-[3-Fluoro-4-[4-(5-nitro-2-thienyl)-1-piperazinyl]phenyl]-2-oxo-5-oxazolidinyl]methyl]fluoroacetamide (Compound No. 11)
(S)—N-[[3-[3-Fluoro-4-[3-(1α,5α,6α)-[6-{N-(5-nitro-2-thienyl)-N-methyl}aminomethyl]-3-azabicyclo-[3.1.0]hexane]phenyl]-2-oxo-5-oxazolidinyl]methyl]acetamide (Compound No 12).
(S)—N-[[3-[3-Fluoro-4-[4-(5-nitro-2-thienyl)-1-homopiperazinyl]phenyl]-2-oxo-5-oxazolidnyl]methyl]acetamide (Compound No.13)
(S)—N-[[3-[3-Fluoro-4-[4-(5-nitro-2-furyl)-1-homopiperazinyl]phenyl]-2-oxo-5-oxazolidinyl]methyl]acetamide (Compound No.14)
(S)—N-[[3-[3-Fluoro-4-[4-{3-thienyl(2-nitro)5-formyl}-1-piperazinyl]phenyl]-2-oxo-5-oxazolidinyl]-methyl]acetamide (Compound No. 15)
(S)—N-[[3-[3-Fluoro-4-[N-1-[4-{N-methyl-N-(5-nitro-2-furyl)}amino]-1-piperadinyl]phenyl]-2-oxo-5-oxazolidinyl]methyl]acetamide (Compound No. 16)
(S)—N-[[3-[3-Fluoro-4-[3-(1α,5α,6α)-[6-{N-(5-nitro-2-furyl)-N-methy}aminomethyl]-3-azabicyclo[3.1.0]hexane]phenyl]-2-oxo-5-oxazolidinyl]methyl]acetamide (Compound No. 17)

5. A pharmaceutical composition comprising the compound of claims 1, 2, 3 or 4 and a pharmaceutical acceptable carrier.

6. A pharmaceutical composition comprising a pharmaceutically effective amount of compound according to claims 1, 2, 3 or 4, or a physiologically acceptable acid addition salt thereof with a pharmaceutical acceptable carrier for treating microbial infections.

7. A method of treating or preventing microbial infections in a mammal comprising administering to said mammal, the pharmaceutical composition according to claim 6.

8. The method according to claim 7 wherein the microbial infections are caused by gram-positive and gram-negative bacteria.

9. The method according to claim 8 wherein the gram-positive bacteria are selected from the group consisting of staphylococcus spp., streptococuus spp., bacillus spp., corynebacterum spp., clostridia spp., peptostreptococus spp., listeria spp. and legionella spp.

10. A method of treating or preventing aerobic and anaerobic bacterial infections in a mammal comprising administering to said mammal, a therapeutically effective amount of a compound having the structure of Formula I 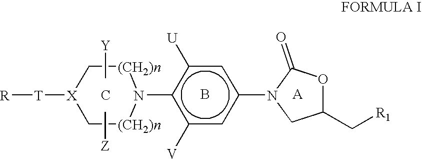
and its pharmaceutically acceptable salts, solvates, polymorphs, enantiomers, diastereomers, N-oxides, prodrugs or metabolites, wherein
T is five membered (un)substituted heterocyclic ring with exclusively one heteroatom, selected from oxygen, nitrogen and sulphur; aryl, substituted aryl, bound to the ring C including aryl and five membered heteroaryl which are further substituted by a group represented by R, wherein R is selected from the group consisting of H, CHO, C₁₋₆ alkyl, F, Cl, Br, I, —CN, COR₅, COOR₅, N(R₆,R₇), NHCOC(R₈,R₉,R₁₀), NHCOOR₁₀, CON (R₆,R₇), CH₂NO₂, NO₂, CH(OAc)₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₄ and R₅ are independently selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy, R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3;
X is C, CH, CH—S, CH—O, N, CHNR₁₁, CHCH₂NR₁₁, CCH₂NR₁₁, wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Y and Z are independently selected from hydrogen, C₁₋₆ alkyl, C₃₋₁₂ and cycloalkyl C₀₋₃ bridging groups;
U and V are independently selected from hydrogen, optionally substituted C₁₋₆ alkyl, F, Cl, Br, C₁₋₁₂ alkyl substituted with one or more of F, Cl, Br, I, preferably U and V are hydrogen or fluoro;
R₁ is selected from the group consisting of —NHC(═O)R₂, N(R₃, R₄), —NR₂C(═S) R₃, —NR₂C(═S)SR₃, wherein R₂ is hydrogen, C₁₋₁₂ alkyl, C₃₋₁₂ cycloalkyl, C₁₋₆ alkoxy, C₁₋₆ alkyl substituted with one or more of F, Cl, Br, I or OH; R₃, R₄ are independently selected from hydrogen, C₁₋₁₂ alkyl, C₃₋₁₂ cycloalkyl, C₁₋₆ alkoxy, C₁₋₆ alkyl substituted with one or more of F, Cl, Br, I or OH.

11. A method of treating or preventing aerobic and anaerobic bacterial infections in a mammal comprising administering to said mammal, a therapeutically effective amount of a compound having the structure of Formula II: 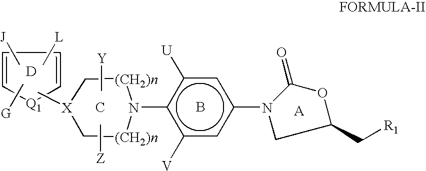
and its pharmaceutically acceptbale salts, solvates, polymorphs, enantiomers, diastereomers, N-oxides, prodrugs or metabolites, wherein
R₁ is selected from the group consisting of (1) —NHC(═O)R₂; (2) —N(R₃, R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or more of F, Cl, Br, I, OH;
U and V are independently selected from hydrogen, optionally substituted C₁₋₆ alkyl, F, Cl, Br, C₁₋₁₂ alkyl substituted with one or more of F, Cl, Br, I;
Y and Z are independently selected from (1) hydrogen, (2) C₁₋₆ alkyl, (3) C₃₋₁₂ cycloalkyl (4) C₀₋₃ bridging group;
X is selected from C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Q₁ is selected from O, S, NR₁₁, wherein R₁₁ is as defined above;
G, J, L are independently selected from H, C₁₋₆ alkyl, F, Cl, Br, I, —CN, COR₅, COOR₅, N(R₆,R₇), NHCOC(R₈,R₉,R₁₀), CON (R₆,R₇), NHCOOR₁₀, CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, L OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl.

12. The method of treating or preventing aerobic and anaerobic bacterial infections of claim 11, wherein ring C is 6-8 membered in size or of larger size and the larger rings have either two or three carbons between each nitrogen atom, comprising of: 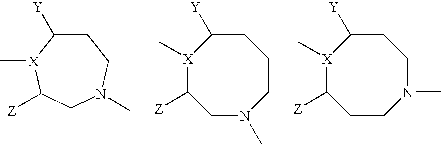
and may be bridged to form a bicyclic system as shown below, 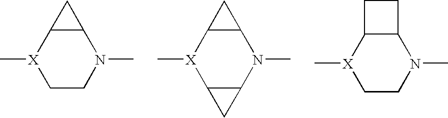
ring C optionally substituted at positions Y and Z with alkyl groups, cycloalkyl groups, fluoro group, carboxylic and corresponding esters, amides, substituted alkyls or bridging alkyl groups as shown below: 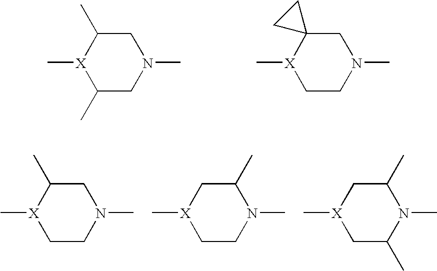
or ring C is 6 membered in size and X is —CH—(NHR₁₁), or >CCH₂NHR₁₁— which is selected from the group consisting of the following rings wherein R₁₁ is the same as defined earlier, 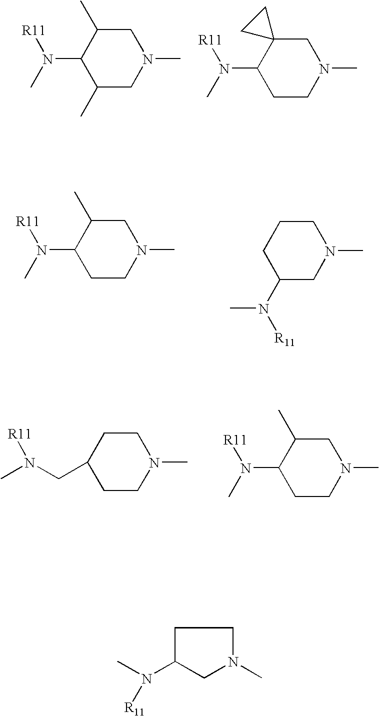
or
in addition to the above, ring C includes the following structures: 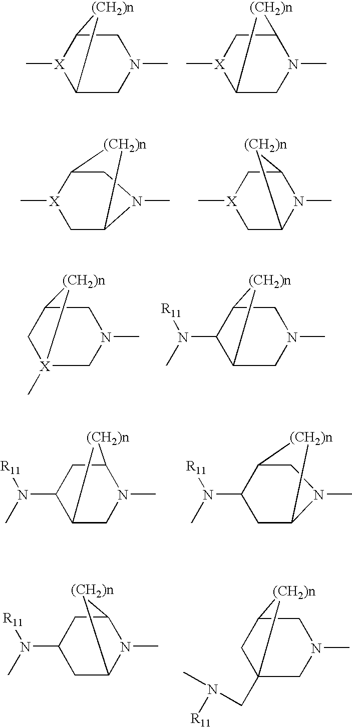
when Q₁=NR₁₁, O or S, the structures are represented by Formulae III, IV and V, respectively, 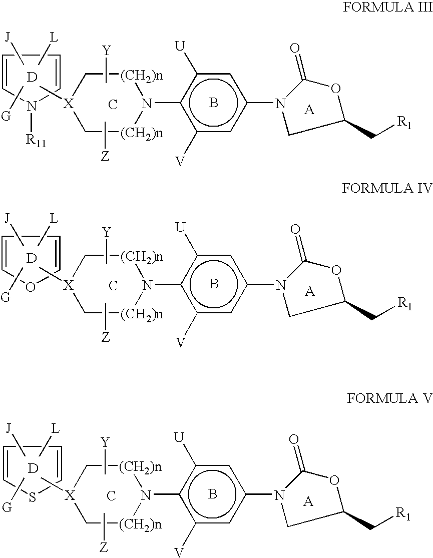
wherein R₁, R₁₁, U, V, X, Y, Z, G, J, L and n in Formula III, Formula IV and Formula V are the same as defined earlier for Formula II.

13. A method of treating or preventing catheter infections and foreign body or prostheses infections in a mammal comprising administering to said mammal, a therapeutically effective amount of a compound having the structure of Formula I. 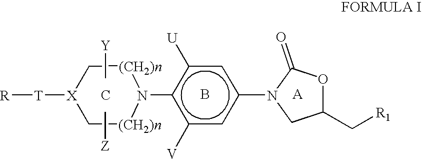
and its pharmaceutically acceptable salts, solvates, polymorphs, enantiomers, diastereomers, N-oxides, prodrugs or metabolites, wherein
T is five membered (un)substituted heterocyclic ring with exclusively one heteroatom, selected from oxygen, nitrogen and sulphur; aryl, substituted aryl, bound to the ring C including aryl and five membered heteroaryl which are further substituted by a group represented by R, wherein R is selected from the group consisting of H, CHO, C₁₋₆ alkyl, F, Cl, Br, I, —CN, COR₅,COOR₅, N(R₆,R₇), NHCOC(R₈,R₉,R₁₀), NHCOOR₁₀, CON(R₆,R₇), CH₂NO₂, NO₂, CH(OAc)₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₄ and R₅ are independently selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3;
X is C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Y and Z are independently selected from hydrogen, C₁₋₆ alkyl, C₃₋₁₂ and cycloalkyl C₀₋₃ bridging groups;
U and V are independently selected from hydrogen, optionally substituted C₁₋₆ alkyl, F, Cl, Br, C₁₋₁₂ alkyl substituted with one or more of F, Cl, Br, I, preferably U and V are hydrogen or fluoro;
R₁ is selected from the group consisting of —NHC(═O)R₂, N(R₃, R₄), NR₂C(═S) R₃, —NR₂C(═S)SR₃, wherein R₂ is hydrogen, C₁₋₁₂ alkyl, C₃₋₁₂ cycloalkyl, C₁₋₆ alkoxy, C₁₋₆ alkyl substituted with one or more of F, Cl, Br, I or OH; R₃,R₄ are independently selected from hydrogen, C₁₋₁₂ alkyl, C₃₋₁₂ cycloalkyl, C₁₋₆ alkoxy, C₁₋₆ alkyl substituted with one or more of F, Cl, Br, I or OH.

14. A method of treating or preventing catheter infections and foreign body or prothesis infections in a mammal comprising administering to said mammal, a therapeutically effective amount of a compound having the structure of Formula II: 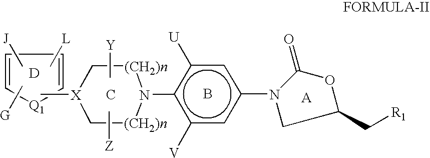
and its pharmaceutically acceptbale salts, solvates, polymorphs, enantiomers, diastereomers, N-oxides, prodrugs or metabolites, wherein
R₁ is selected from the group consisting of (1) —NHC(═O)R₂; (2) —N(R₃, R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆alkyl substituted one or more of F, Cl, Br, I, OH;
U and V are independently selected from hydrogen, optionally substituted C₁₋₆ alkyl, F, Cl, Br, C₁₋₁₂ alkyl substituted with one or more of F, Cl, Br, I;
Y and Z are independently selected from (1) hydrogen, (2) C₁₋₆ alkyl, (3) C₃₋₁₂ cycloalkyl (4) C₀₋₃ bridging group;
X is selected from C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Q₁ is selected from O, S, NR₁₁, wherein R₁₁ is as defined above;
G, J, L are independently selected from H, C₁₋₆ alkyl, F, Cl, Br, I, —CN, COR₅,COOR₅, N(R₆,R₇), NHCOC(R₈,R₉,R₁₀), CON (R₆,R₇), NHCOOR₁₀, CH₂NO₂, NO₂, CH₂R₉, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl or heteroaryl.

15. A method of treating or preventing catheter infections and foreign body or prothesis infections in a mammal comprising administering to said mammal, a therapeutically effective amount of a compound having the structure of Formula II as defined in claim 14 wherein ring C is 6-8 membered in size or of larger size and the larger rings have either two or three carbons between each nitrogen atom, comprising of: 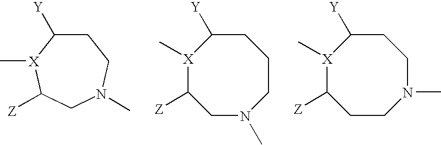
and may be bridged to form a bicyclic system as shown below, 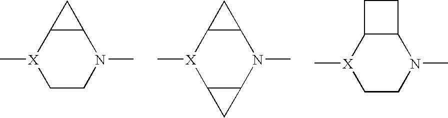
ring C optionally substituted at positions Y arid Z with alkyl groups, cycloalkyl groups, fluoro group, carboxylic and corresponding esters, amides, substituted alkyls or bridging alkyl groups as shown below: 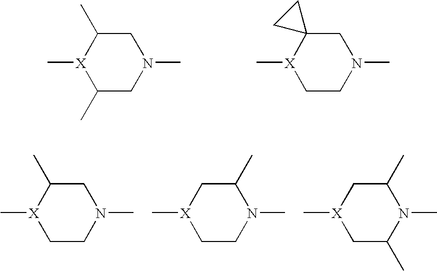
or ring C is 6 membered in size and X is —CH—(NHR₁₁), or >CCH₂NHR₁₁— which is selected from the group consisting of the following rings wherein R₁₁ is the same as defined earlier, 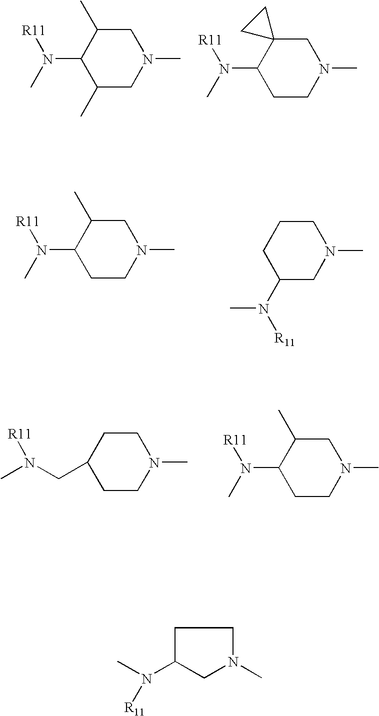
or
in addition to the above, ring C includes the following structures: 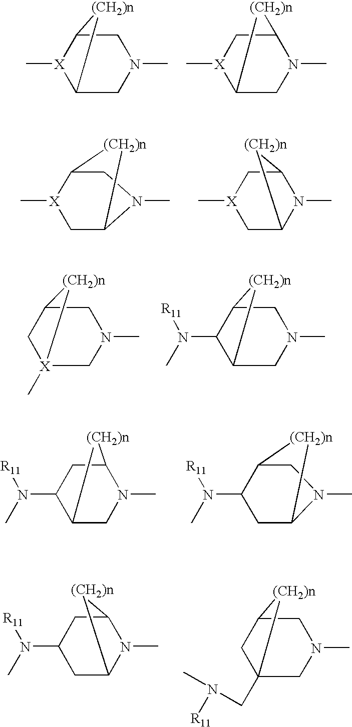
when Q₁=NR₁₁, O or S, the structures are represented by Formulae III, IV and V, respectively, 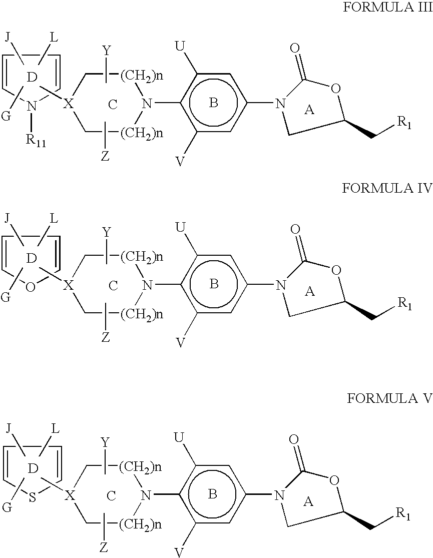
wherein R₁, R₁₁, U, V, X, Y, Z, G, J, L and n in Formula III, Formula IV and Formula V are the same as defined earlier for Formula II.

16. A process for preparing a compound of Formula I 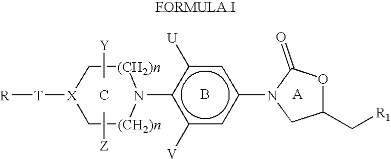
and its pharmaceutically acceptable salts, solvates, polymorphs, enantiomers, diastereomers, N-oxides, prodrugs or metabolites. wherein
T is five membered (un)substituted heterocyclic ring with exclusively one heteroatom, selected from oxygen, nitrogen and sulphur; aryl, substituted aryl, bound to the ring C including aryl and five membered heteroaryl which are further substituted by a group represented by R, wherein R is selected from the group consisting of H, CHO, C₁₋₆ alkyl, F, Cl, Br, I, —CN, COR₅, COOR₅, N(R₆, R₇), NHCOC(R₈, R₉, R₁₀), NHCOOR₁₀, CON (R₆, R₇), CH₂NO₂, NO₂, CH(OAc)₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more of F, Cl, Br, I, OR₄, SR₄; wherein R₄ and R₅ are independently selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ alkyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
n is an integer in the range from 0 to 3;
X is C, CH, CH—S, CH—O, N, CHNR₁₁, CHCH₂NR₁₁, CCH₂NR₁₁; wherein R₁₁ is hydrogen, optionally substituted C₁₋₁₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Y and Z are independently selected from hydrogen, C₁₋₆ alkyl, C₃₋₁₂ and cycloalkyl C₀₋₃ bridging groups;
U and V are independently selected from hydrogen, optionally substituted C₁₋₆ alkyl, F, Cl, Br, C₁₋₂ alkyl substituted with one or more of F, Cl, Br, I, preferably U and V are hydrogen or fluoro;
R₁ is selected from the group consisting of —NHC(═O)R₂, N(R₃,R₄), —NR₂C(═S), R₃, —NR₂C(═S)SR₃, wherein R₂ is hydrogen, C₁₋₁₂ alkyl, C₃₋₁₂ cycloalkyl, C₁₋₆ alkoxy, C₁₋₆ alkyl substituted with one or more of F, Cl, Br, I or OH; R₃,R₄ are independently selected from hydrogen, C₁₋₁₂ alkyl, C₃₋₁₂ cycloalkyl, C₁₋₆ alkoxy, C₁₋₆ alkyl substituted with one or more of F, Cl, Br, I or OH;
which comprises reacting an amine of Formula VI 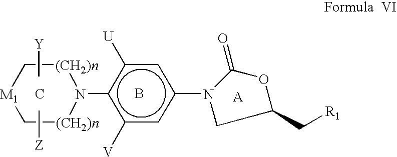
with a heteroaromatic compound of Formula R-T-R₁₂ wherein T, R₁, Y, Z, U, V and n are the same as defined earlier and M₁ is selected from the group consisting of NH, NHR, CHNHR, —CHCH₂NHR, —CCH₂NHR wherein R is H, ethyl, methyl, isopropyl, acetyl, cyclopropyl, alkoxy or acetyl and R₁₂ is a suitable leaving group selected from the group consisting of fluoro, chloro, bromo, iodo, SCH₃, —SO₂CH₃, —SO₂CF₃, Tos and OC₆H₅.

17. The process of claim 16, wherein the amine of Formula VI reacts with a heteroaromatic compound of Formula R-T-R₁₂ in the presence of a base selected from the group consisting of potassium carbonate, N-ethyldiisopropylamine and dipotassium hydrogenphosphate.

18. A process for preparing a compound of Formula II 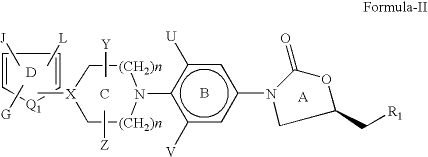
and its pharmaceutically acceptbale salts, solvates, polymorphs, enantiomers, diastereomers, N-oxides, prodrugs or metabolites, wherein
R₁ is selected from the group consisting of (1) —NHC(═O)R₂; (2) —N(R₃, R₄); (3) —NR₂C(═S)R₃; (4) —NR₂C(═S)SR₃ wherein R₂, R₃, R₄ are independently hydrogen, C₁₋₁₂ alkyl, C₃₋₁₂ cycloalkyl, C₁₋₆ alkoxy, C₁₋₆ alkyl substituted one or more of F, Cl, Br, I, OH;
U and V are independently selected from hydrogen, optionally substituted C₁₋₆ alkyl, F, Cl, Br, C₁₋₁₂ alkyl substituted with one or more of F, Cl, Br, I;
Y and Z are independently selected from (1) hydrogen, (2) C₁₋₆ alkyl, (3) C₃₋₁₂ cycloalkyl (4) C₀₋₃ bridging group;
X is selected from C, CH, CH—S, CH—O, N, CHNR₁₁, CHCH₂NR₁₁, CCH₂NR₁₁; wherein R₁₁ is hydrogen, optionally substituted C₁₋₂ alkyl, C₃₋₁₂ cycloalkyl, C₁₋₆ alkoxy, C₁₋₆ alkyl, C₁₋₆ alkylcarbonyl, C₁₋₆ alkylcarboxy, aryl, heteroaryl;
Q₁ is selected from O, S, NR₁₁, wherein R₁₁ is as defined above;
G, J, L are independently selected from H, C₁₋₆ alkyl, F, Cl, Br, I, —CN, COR₅, COOR₅; N(R₆,R₇), NHCOC(R₈,R₉,R₁₀), CON (R₆,R₇), NHCOOR₁₀, CH₂NO₂, NO₂, CH₂R₈, CHR₉, —CH═N—OR₁₀, —C═CH—R₅, OR₅, SR₅, —C(R₉)═C(R₉)NO₂, C₁₋₁₂ alkyl substituted with one or more F, Cl, Br, I, OR₄, SR₄; wherein R₅ is selected from H, C₁₋₁₂ alkyl, C₃₋₁₂ cycloalkyl, C₁₋₆ alkoxy, C₁₋₆ alkyl substituted with one or more of F, Cl, Br, I or OH, aryl, heteroaryl; R₆ and R₇, are independently selected from H, optionally substituted C₁₋₁₂ allyl, C₃₋₁₂ cycloalkyl, C₁₋₆ alkoxy; R₈ and R₉ are independently selected from H, C₁₋₆ alkyl, F, Cl, Br, I, C₁₋₁₂ alkyl substituted with one or more of F, Cl, Br, I, OR₅, SR₅, N(R₆,R₇); R₁₀═H, optionally substituted C₁₋₁₂ alkyl, C₃₋₁₂ cycloalkyl, C₁₋₆ alkoxy, C₁₋₆ alkyl, aryl, heteroaryl;
comprising reacting a compound of Formula VI 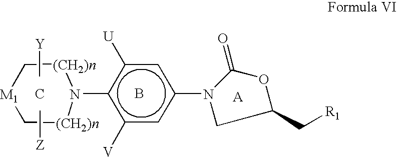
with a heteroaromatic compound of Formula VII 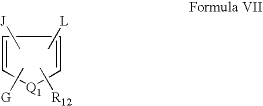
wherein R₁, Y, Z, U, V, G, J, L, Q₁, and n are the same as defined earlier and M₁ is selected from the group consisting of NH, NHR, CHN, —CHCH₂NHR, —CCH₂NHR wherein R is H, ethyl, methyl, isopropyl, acetyl, cyclopropyl, alkoxy or acetyl and R₁₂ is a suitable leaving group selected from the group consisting of fluoro, chloro, bromo, iodo, SCH₃, —SO₂CH₃, —SO₂CF₃, Tos and OC₆H₅.

19. The process for preparing a compound of Formula II as described in claim 18 wherein ring C in Formula II is 6-8 membered in size or of larger size and the larger rings have either two or three carbons between each nitrogen atom, comprising of: 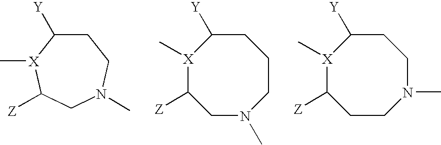
and may be bridged to form a bicyclic system as shown below, 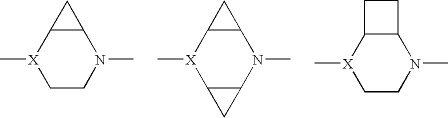
ring C optionally substituted at positions Y and Z with alkyl groups, cycloalkyl groups, fluoro group, carboxylic and corresponding esters, amides, substituted alkyls or bridging alkyl groups as shown below: 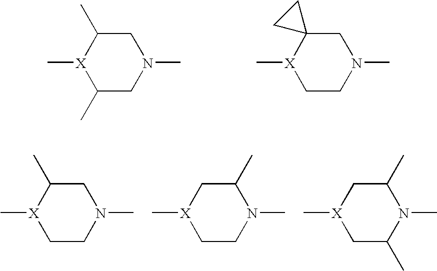
or ring C is 6 membered in size and X is —CH—(NH₁₁), or >CCH₂NHR₁₁— which is selected from the group consisting of the following rings wherein R₁₁ is the same as defined earlier, 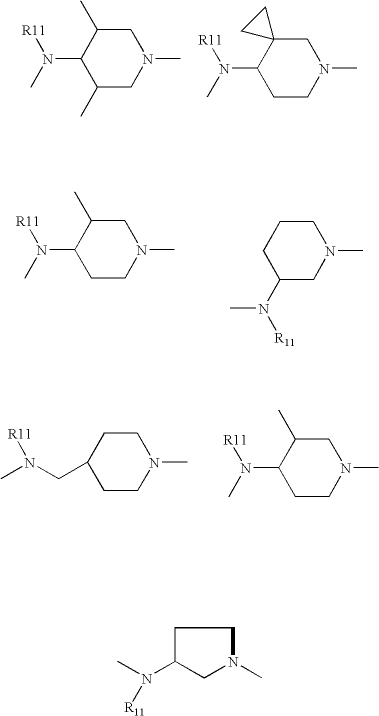
or
in addition to the above, ring C includes the following structures: 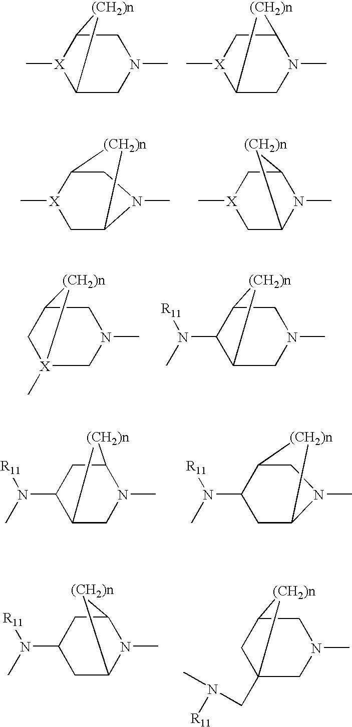
when Q₁=NR₁₁, O or S, the structures are represented by Formulae III, IV and V, respectively, 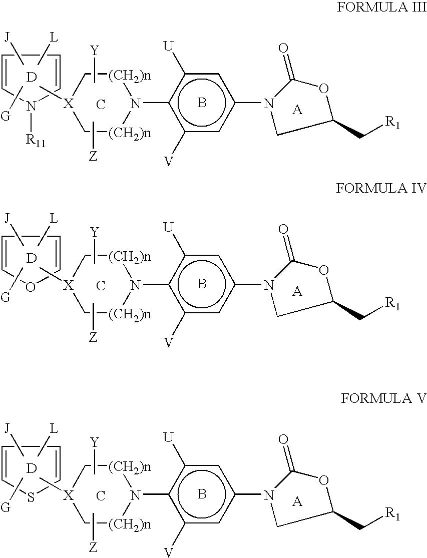
wherein R₁, R₁₁, U, V, X, Y, Z, G, J, L and n in Formula III, Formula IV and Formula V are the same as defined earlier for Formula II.

20. The process of claim 18 wherein the heteroaromatic compound of Formula VII is reacted with the amine of Formula VI in the presence of ligands selected from the group consisting of Pd₂(dba)₃ and Pd(OAc)₂.

21. The process of claim 18 wherein the heteroaromatic compound of Formula VII is 2-bromothiophene.

22. The process of claim 18 wherein the reaction of compound of Formula VI with a compound of Formula VII is carried out in the presence of a solvent wherein the solvent is selected from the group consisting of dimethylformamide, dimethylacetamide, acetanitrile, dimethylsulfoxide and ethylene glycol.

23. The process of claim 18 wherein the reaction of compound of Formula VI with a compound of Formula VII is carried out in the presence of a suitable base wherein the base is selected from the group consisting of triethylamine diisopropylethylamine, potassium carbonate, sodium carbonate and dipotassium hydrogen phosphate.
